(19) United States
(12) Patent Application Publication
(10) Pub. No.: US 2021/0238580 A1
(43) Pub. Date: Aug. 5, 2021

(54) LYSIS METHOD FOR PLANT SAMPLES

(71) Applicant: QIAGEN GmbH, Hilden (DE)

(72) Inventors: Dominic O'NEIL, Hilden (DE); Patrick SMITH, Hilden (DE)

(21) Appl. No.: 17/051,310

(22) PCT Filed: Apr. 29, 2019
(86) PCT No.: PCT/EP2019/060983

§ 371 Date: Oct. 28, 2020

(30) Foreign Application Priority Data
May 11, 2018 (EP) 18171964.2
Aug. 16, 2018 (EP) 18189422.1

(51) Int. Cl. C12N 15/10; G01N 1/40; G01N 1/28; C12Q 1/6806

(52) U.S. Cl. CPC C12N 15/1003; C12Q 1/6806; G01N 1/286; G01N 1/4055

(57) ABSTRACT

The present invention provides a lysis method for releasing microbial nucleic acids from microorganisms comprised in a plant sample, comprising mechanically disrupting the plant sample in a liquid lysis composition using at least two types of solid disrupting particles, wherein (i) the first type is provided by one or more disrupting particles having a size of at least 1.5 mm and (ii) the second type is provided by a plurality of disrupting particles having a size of 1 mm or less. Also provides is a method for isolating nucleic acids including microbial nucleic acids from a plant sample and kits.

### BACKGROUND OF THE INVENTION

Nucleic acid isolation from plants can be very challenging due to the difficult to lyse nature of plant cells and the presence of large amounts of inhibitors, including polysaccharides and polyphenolic compounds. In addition these parameters can vary dramatically between plant types and different parts of the same plant. This often leads to the isolation of low amount of poor quality of nucleic acids such as DNA.

The most often used methods of lysis for plant samples is either a mortar and pestle or mechanical disruption with common grinding media (i.e., metal or glass beads). When such standard procedures are used, yields are typically low and the DNA includes the presence of large amounts of inhibitors depending on the sample type.

For isolating nucleic acids such as DNA from plants a commonly used method uses a mortar and pestle in combination with a CTAB lysis buffer for both lysis of plant material and removal of inhibitors. The use of a mortar and pestle is time consuming, ineffective, and difficult to use with multiple samples. CTAB is a toxic agent. Commercially available kits use either a mortar and pestle or some combination of spherically shaped ceramic or metal beads combined with chaotropic buffers and detergent based buffers for lysis. The disadvantages of these approaches are that they are non-standard in nature and cannot be applied to multiple different plant types with similar success. This often leads to lower yields, increased presence of inhibitors and, in the case of mortar and pestle, time-consuming experiments.

Furthermore, there is a need for methods that allow to efficiently isolate also microbial nucleic acids from plant samples. Many plant samples comprise microorganisms (e.g. on leafs or within the plant tissue). There is a need to make microbial nucleic acids comprised in plant samples available for analysis. With the rise of next generation sequencing (NGS) and in particular microbiome research (bacteria/fungi/virus) the interest in the isolation of high quality DNA that can be immediately used for these applications is growing exponentially. This is also true for the field of plant biology. A major challenge when aiming at isolating microbial nucleic acids comprised in plant samples is the mixture of plant host cells in combination with the target microbial cells. The difficult to lyse nature and presence of high amounts of PCR and enzymatic inhibitors makes the use of common prior art methods less effective.

There is a need for improved methods for isolating nucleic acids, such as in particular DNA, from plant samples.

In particular, methods are needed that also allow to efficiently release microbial nucleic acids from microorganisms comprised in plant samples and thus microorganisms that exists on, around or within the plant sample itself so that these microbial nucleic acids can be isolated from the obtained lysate. In particular, there is a need is for improved lysis methods that efficiently release nucleic acids, such as DNA, from a plant sample, while also efficiently releasing microbial nucleic acids, such as microbial DNA, from microorganisms comprised in the plant sample to make them available for subsequent isolation. In particular, there is the need for improved lysis and isolation methods that increase the amount of microbial nucleic acids, such as microbial DNA from different plant samples, such as in particular various plant roots.

Furthermore, there is a need for a protocol that increases the nucleic acid yield and inhibitor removing for a large variety of plant sample types.

It is the object of the present invention to overcome at least one drawback of the prior art. In particular, it is an object of the present invention to provide a method that meets at least one of the needs. It is also an object of the present invention to provide an improved lysis method that efficiently releases nucleic acids, such as DNA, from various plant samples, that also efficiently releases microbial nucleic acids, such as microbial DNA, from microorganisms that are comprised in the plant sample to make them available for subsequent isolation.

### SUMMARY OF THE INVENTION

According to a first aspect, a lysis method for releasing microbial nucleic acids from microorganisms comprised in a plant sample is provided, comprising mechanically disrupting the plant sample in a liquid lysis composition using at least two types of solid disrupting particles, wherein

(i) the first type is provided by one or more disrupting particles having a size of at least 1.5 mm and
(ii) the second type is provided by a plurality of disrupting particles having a size of 1 mm or less.

According to a second aspect a method for isolating nucleic acids including microbial nucleic acids from a plant sample is provided, comprising

(a) performing the lysis method according to the first aspect;
(b) isolating nucleic acids from the lysed and optionally further processed sample; and
(c) optionally sequencing isolated nucleic acid, preferably sequencing isolated DNA.

According to a third aspect, a lysis system, preferably a kit, is provided for releasing microbial nucleic acids from microorganisms comprised in a plant sample, comprising

(a) a liquid lysis composition
(b) at least two types of solid disrupting particles, wherein
(i) the first type is provided by one or more disrupting particles having a size of at least 1.5 mm; and
(ii) the second type is provided by a plurality of disrupting particles having a size of less than 1 mm.

The present invention is also directed to the use of such lysis system for lysing plant samples comprising or suspected of comprising microorganisms.

According to a forth aspect, the present disclosure pertains to the use of the system according to the third aspect in a method according to the first aspect.

According to a fifth aspect, the present disclosure pertains to the use of the system according to the third aspect for lysing a plant sample and releasing microbial nucleic acids from microorganisms comprised in the plant sample, wherein the user may use (i) the first and the second type or (ii) the second type of disrupting particles for plant sample lysis to release microbial nucleic acids, preferably DNA, from microorganisms comprised in the plant sample.

The use of the first type of disrupting particle as disclosed herein provides high DNA yields and therefore is particularly effective in homogenizing and hence disrupting various plant samples. However, such large disrupting particle alone is less effective in mechanically disrupting microorganisms such as bacteria that are comprised in the plant sample. The combined use of at least one particle according to the first type such as e.g. a ballcone together with a plurality of smaller particles (e.g. zirconium beads) as second type achieves a high total yield of isolated total DNA. Moreover, the combined use of the first and second type of particles efficiently releases microbial nucleic acids comprised in microbes comprised in plant samples, as is shown by the high percentage of bacterial DNA among total DNA isolated (see examples). Therefore, the combined use of the first and second type of disrupting particles as taught herein, especially when being used in conjunction with the lysis chemistry disclosed herein, is preferred. The present invention can be advantageously used to release nucleic acids including microbial nucleic acids from various plant samples, including difficult to lyse samples such as root samples.

The combined mechanical lysis described herein provides DNA from various plant samples with high yield, wherein the obtained DNA comprises a high amount of microbial DNA which is therefore available for analysis. For analysis, various methods can be used, such as amplification based procedures (for example PCR) as well as sequencing, such as next generation sequencing.

Other aspects, objects, features, and advantages of the present application will become apparent to those skilled in the art from the following description and appended claims. It should be understood, however, that the following description, appended claims, and specific examples, while indicating preferred embodiments of the application, are given by way of illustration only.

In the following description, any ranges provided herein include all the values in the ranges.

It should also be noted that the term “or” is generally employed in its sense including “and/or” (i.e., to mean either one, both, or any combination thereof of the alternatives) unless the content dictates otherwise.

Also, as used in this specification and the appended claims, the singular forms “a,” “an,” and “the” include plural referents unless the content dictates otherwise.

The terms “include,” “have,” “comprise” and their variants are used synonymously and to be construed as non-limiting.

The term “a combination thereof” as used herein refers to one of the all possible combinations of the listed items preceding the term. For example, “A, B, C, or a combination thereof” is intended to refer to any one of: A, B, C, AB, AC, BC, or ABC. Similarly, the term “combinations thereof” as used herein refers to all possible combinations of the listed items preceding the term. For instance, “A, B, C, and combinations thereof” is intended to refer to all of: A, B, C, AB, AC, BC, and ABC.

### DETAILED DESCRIPTION OF THE INVENTION

The present invention provides an improved lysis method for lysing plant samples that efficiently releases microbial nucleic acids from microorganisms comprised in various plant samples. The overall yield is high and the released nucleic acids comprise a high amount of microbial nucleic acids that are therefore available for subsequent isolation and analysis. The technology disclosed herein allows to obtain DNA from plant samples with high yield, wherein the obtained DNA not only comprises plant DNA with a good yield, but furthermore comprises a high amount of microbial DNA which therefore becomes available for analysis.

Furthermore, methods are described that allow to isolate nucleic acids from a plant sample while inhibitors are removed from the isolated nucleic acids, allowing effective downstream analysis of isolated nucleic acids.

### The Method According to the First Aspect

According to a first aspect, a lysis method for releasing microbial nucleic acids from microorganisms comprised in a plant sample is provided, comprising mechanically disrupting the plant sample in a liquid lysis composition using at least two types of solid disrupting particles, wherein

(i) the first type is provided by one or more disrupting particles having a size of at least 1.5 mm and
(ii) the second type is provided by a plurality of disrupting particles having a size of 1 mm or less.

The individual steps and preferred embodiments will now be described in detail.

The method according to the present disclosure mechanically disrupts the plant sample in a liquid lysis composition. For mechanical disruption, at least two types of solid disrupting particles are used. The disrupting particles are agitated, e.g. mixed or vortexed, as is further described herein so that disruptive forces are applied to the plant sample and comprised microorganisms upon contact. The plant sample is preferably homogenized.

The first type and the second type of solid disrupting particles preferably differ from each other not only is size but also in shape and/or material. Preferably, the one or more disrupting particle used as first type is not spherical and has at least one discontinuity, preferably an edge, and the plurality of particles used as second type are substantially spherical.

### The First Type of Solid Disrupting Particles

For mechanical disruption of the plant sample, at least one solid disrupting particle is used as first type.

The at least one disrupting particle that is used as first type is non-spherical and preferably has an irregular shape. It is an important advantage that the one or more disrupting particles used as first type are non-spherical.

In a particularly preferred embodiment, the surface of the one or more disrupting particle used as first type has at least one discontinuity, especially preferred an edge or a peak. According to one embodiment, the disrupting particle used has one or more beveled edges. The discontinuity provides the advantage that it can be used to apply an irregular, preferably point- or line-impact onto the plant material to be disrupted. It also allows the contact between the particle and the plant material to be of a random nature. The discontinuity leads to movements of the particles that are more irregular compared to movements of spherical-shaped particles and can be used to more randomly attack the plant material to be disrupted. The disruptive forces are increased where the discontinuity, such as an edge or peak, hits the plant material to be disrupted. This allows to efficiently disrupt even very diverse plant materials and thereby allows the disruption of many different plant sample types. As disclosed, a single disrupting particle of this kind may be used as first type.

In a preferred embodiment the surface of the particle contains a first part and contains a second part, whereby the first part and the second part meet by forming an edge. Preferably, the edge extends along a line. The line can be a circle or an arc. The line could also be a straight line. In a preferred embodiment, the first part is the surface of a frustum of a cone and the second part is the surface of a frustum of a cone. Preferably in the embodiment where the first part is the surface of a frustum of a cone and the second part is the surface of a frustum of a cone, both cones are set against each other with their larger base, the edge being formed, where the larger bases meet, the larger basis preferably being of the same diameter. The particle preferably has one, in a preferred embodiment only one line of symmetry. Preferably the particle is of rotational symmetry about the line of symmetry. The edge may be provided in the form of sloping central flange, as is e.g. illustrated in FIGS. 6 to 8.

The one or more particles used as first type may have a subportion that is made up of a section or a part of a ball or an ellipse.

The particle may have a tip. In one embodiment, the tip is a frustum of a cone. According to one embodiment, the larger base of the frustum of the cone that provides the tip is set against the smaller base of the frustum of the cone of the second part. In this embodiment, a subportion that is made up of a section or a part of a ball or an ellipse may be set against the smaller base of the frustum of the cone of the first part. In one embodiment, the subportion that is made up of a section or a part of a ball or an ellipse is a semi-sphere. An embodiment of such disrupting particle, which preferably is a ballcone, is shown in FIG. 6. At least of such particle can be used as first type.

In one embodiment, the particle comprises at least two tips, wherein preferably, both tips are a frustum of a cone. According to one embodiment, the larger base of the frustum of a cone that provides the first tip is set against the smaller base of the frustum of the cone of the first part and the larger base of the frustum of a cone that provides the second tip is set against the smaller base of the frustum of the cone of the second part. An embodiment of such particle with two tips is shown in FIG. 7. At least of such particle can be used as first type.

In one embodiment, the particle has two subportions, wherein each subportion is made up of a section or a part of a ball or an ellipse. In this embodiment, the first subportion that is made up of a section or a part of a ball or an ellipse is set against the smaller base of the frustum of the cone of the first part and the second subportion that is made up of a section or a part of a ball or an ellipse is set against the smaller base of the frustum of the cone of the second part. An embodiment of such particle with two semi-spheres is illustrated in FIG. 8. At least of such particle can be used as first type.

The non-spherical particles used as first type for disruption may have one or more discontinuities such as edges. They may be in the form of solid cones, cylinders, cubes, triangles, rectangles and similar suitable geometric forms. A further example is a diagonal with beveled edges. According to one embodiment, the at least one disrupting particle used as first type has an irregular shape, and may be selected from a ballcone and satellite (shaped like Saturn, planet or UFO), for effecting a disruption of the plant tissue material when mixing or milling forces are applied to the plant sample in the composition of the present invention. The use of a ballcone is particularly effective for disrupting plant samples and therefore is preferred. The solid disrupting particles should be selected in view of not deteriorating or disrupting the released cellular components or analytes.

To achieve sufficient disruption and homogenization of the plant material and undamaged liberation of the desired nucleic acids for isolation, it is preferred that the one or more solid disrupting particles that are used as first type are solid inert particles, i.e. particles made of a material, which does not react with the tissue material, with any of the reagents of the composition and in any case not with the desired nucleic acids to be liberated upon disruption. It is particularly preferred that the released nucleic acids cannot adsorb or adhere to the inert solid disrupting particles used as first type under the used lysis conditions. Suitable inert materials comprise for example inert metals, steel, stainless steel, plastic, and ceramic. Preferably, the at least one disrupting particle used as first type is made of metal. Preferably, it is made of steel, stainless steel, tungsten or other heavy metals. Further examples are metals and alloys from tantalum, platinum, etc. Steel materials include but are not limited to carbon steel, stainless steel and chrome steel. Steel such as stainless steel is preferred. Further suitable inert disrupting materials are known from commercially available inert disrupting particles. It is also possible to use mixtures of one or more kind of disrupting particles, i.e. use disrupting particles of different forms and/or made of different inert materials as first type.

It is further preferred that the one or more disrupting particles used as first type exhibit a sufficient hardness so that no abrasion occurs during the milling or grinding process.

In one embodiment, the one or more non-spherical disrupting particles used as first type have a density that lies in a range selected from 5.0 g/cc to 20 g/cc, 5.5 g/cc to 15 g/cc, 6 g/cc to 12 g/cc and 6 g/cc to 10 g/cc.

To achieve sufficient disruption forces to effectively disrupt and in particular homogenize the plant sample the disrupting particles used as first type should preferably exhibit a comparably large size. This is advantageous and allows the isolation of nucleic acids such as DNA from various plant sample types with high yield.

The one or more solid disrupting particles used as first type have a size of at least 1.5 mm. They may have a size of at least 2 mm, at least 2.5 mm or at least 3 mm. Further, the one or more disrupting particles used as first type may exhibit a size of at least 3 mm 3 mm), at least 3.5 mm or preferably of at least 4 mm. The at least one solid disrupting particle used as first type may exhibit a size of at least 4.5 mm or at least 5 mm.

The one or more disrupting particles used as first type may exhibit a size of up to 15 mm, e.g. up to 12 mm, up to 10 mm or up to 8 mm.

The one or more disrupting particles used as first type may exhibit a size of 1.5 mm to 15 mm, e.g. 2 mm to 15 mm, 2.5 mm to 15 mm, 3 mm to 15 mm or 4 mm to 15 mm. The particles may further exhibit a size of 1.5 mm to 12 mm, e.g. 2 mm to 12 mm, 2.5 mm to 12 mm, 3 mm to 12 mm or 4 mm to 12 mm. Further, the one or more disrupting particles used as first type may exhibit a size of 1.5 mm to 10 mm, e.g. 2 mm to 10 mm, 2.5 mm to 10 mm, 3 mm to 10 mm, or 4 mm to 10 mm. The one or more disrupting particles used as first type may further exhibit a size of 1.5 mm to 7 mm, e.g. 2 mm to 7 mm, 2.5 mm to 7 mm, 3 mm to 7 mm, 3.5 mm to 7 mm, or 4 mm to 7 mm. Most preferred is a size of 3 mm to 7 mm or of 4 mm to 7 mm. It is also possible to use mixtures of disrupting particles of different sizes in such ranges in case more than one disrupting particle is used as first type.

The defined sizes of the one or more disrupting particles used as first type indicate the longest distance between two opposite points of the respective particle. As discussed, the use of irregularly shaped particles having at least one discontinuity at their surface, such as satellites or ballcones is preferred for use as first type. Here, the longest distance between two opposite points is usually the diameter of the “saturn-like ring” surrounding the ball or ballcone part of such particles.

Depending on the size of the one or more disrupting particles used as first type, one or more disrupting particles can be used. In the case of very large particles, the desired results of disruption and preservation of the analytes may be achieved with only one particle (in particular one ballcone). It is particularly preferred to use one, i.e. a single, disrupting particle as first type. As described herein, the use of a single ballcone is preferred for disrupting the plant sample material.

Examples of commercially available particles of irregular shape, ballcones or satellite-shaped particles, which are preferred, exhibit the following sizes:

TABLE I
Sizes [mm]

| | | | | | | height (top of the | |
|---|---|---|---|---|---|---|---|
| | | | | | | cone to the | |
| ball diameter × | | ball | | ring | | opposite located | |
| ring diameter | | diameter | | diameter | | side of the ball) | |
| 3 × 5 | mm | 3 | mm | 5 | mm | 3.6 | mm |
| 4 × 6 | mm | 4 | mm | 6 | mm | 4.7 | mm |
| 5 × 7 | mm | 5 | mm | 7 | mm | 5.7 | mm |
| 6.5 × 8.5 | mm | 6.5 | mm | 8.5 | mm | 8 | mm |

Therein, one half of the steel ballcone is a semi-sphere (ball), the other half is a cone and both are separated by a sloping central flange (ring). An example of a ballcone is provided in the figures.

As discussed herein, the at least one solid disrupting particle used as first type is preferably a heavy solid device. According to one embodiment, the weight of the solid disrupting particle is at least 300 mg, e.g. at least 400 mg, at least 500 mg, at least 600 mg or at least 700 mg. In embodiments, the weight of the solid disrupting particle lies in the range of 300 mg-1500 mg, 400 mg to 1250 mg, 500 mg to 1000 mg and 600 mg to 900 mg.

A weight of 500 mg to 1000 mg or 600 mg to 900 mg is preferred. This is particularly if a single disrupting particle of irregular shape (e.g. a ballcone) is used as first type for mechanical disruption. A disrupting particle of such weight may further exhibit a size of 1 mm to 10 mm, e.g. 1.5 mm to 9 mm, 2 mm to 8 mm, 2.5 mm to 7 mm, 3 mm to 7 mm or 4 mm to 7 mm. Most preferred is a size 3 mm to 7 mm, preferably 4 mm to 7 mm and a weight of 500 to 1000 mg, preferably 600 mg to 900 mg. The disrupting particle of irregular shape that is used as first type is preferably a ballcone. As discussed herein, the use of a single ballcone is advantageous.

As disclosed, it is preferred to use a single non-spherical disrupting particle (e.g. having a size that lies in the range of 3 mm to 7 mm, preferably 4 mm to 7 mm and a weight of 500 to 1000 mg, preferably 600 mg to 900 mg, such as a single ballcone, as first type.

### The Second Type of Solid Disrupting Particle

The second type of solid disrupting particles that is used in combination with the first type is provided by a plurality of disrupting particles having a size of 1 mm or less. The particles of the second type are therefore smaller than the particles of the first type. In addition, a plurality of such particles is used as second type. The second type of disrupting particles in particular supports the efficient lysis of microorganisms that are comprised in plant sample, such as bacteria and/or fungi that may be present on, around or within the plant sample. As is demonstrated and explained further in the examples, the use of the combination of the first and second type of particles provides high nucleic acids yields, wherein furthermore, the amount of comprised microbial nucleic acid is improved.

The particles of the plurality of particles used as second type are substantially spherical. Conventional beads used in the art are usually described as “substantially” spherical because those beads are not necessarily mathematically-perfect spheres, but may include minor imperfections that affect their shape. As discussed herein, the use of the one or more larger non-spherical disrupting particle as first type (described above, such as e.g. a ballcone or similar) in combination with the plurality of smaller substantially spherical disrupting particles provides particularly advantageous results with respect to overall DNA yield and yield of microbial nucleic acids, such as bacterial DNA.

According to one embodiment, the particles of the plurality of particles that are used as second type are crystalline particles.

According to one embodiment, the plurality of particles used as second type comprise or consist of zirconium, zircon (zirconium silicate), zirconia (zirconium dioxide), yttrium-stabilized zirconium, quartz, aluminum oxide, silicon carbide, ceramic, glasses (e.g. silicon dioxide glass or silica) or a combination of the foregoing. According to one embodiment, the particles of the plurality of particles used as second type are substantially spherical and comprise or consist of zirconium, zircon (zirconium silicate), zirconia (zirconium dioxide) or yttrium-stabilized zirconium. According to one embodiment, the particles of the plurality of particles used as second type are made of the same material.

The particles of the plurality of disrupting particles used as second type are smaller than the first type and have a size of 1 mm or less. The defined sizes of the one or more disrupting particles used as second type indicate the longest distance between two opposite points of the respective particle. As the particles of the second type are substantially spherical, this is the diameter.

According to one embodiment, the plurality of particles used as second type have a size that lies in the range selected from 0.05 mm to 0.9 mm, e.g. 0.07 mm to 0.8 mm, 0.08 mm to 0.75 mm and 0.09 mm to 0.7 mm. As discussed, the particles are preferably spherical. Bead sizes indicated by vendors are usually median (average) values. Because spherical beads are usually sorted by sieves according to their size, the bead size may vary between +/−10% of a listed value. As discussed herein, one may use a plurality of particles having at least two different sizes, wherein, however, the particles used as second type have a size of less than 1 mm and preferably all lie in the defined ranges.

According to one embodiment, the plurality of particles used as second type have at least two different sizes, wherein (i) the first particle size lies on average in a range selected from 0.05 mm to 0.25 mm and (ii) the second particle size lies on average in a range selected from 0.3 mm to 0.9 mm.

According to one embodiment, the plurality of particles used as second type have at least two different sizes, wherein (i) the first particle size lies on average in a range selected from 0.05 mm to 0.25 mm, 0.07 mm to 0.2 mm, 0.08 mm to 0.175 mm and 0.9 mm to 0.15 mm and (ii) the second particle size lies on average in a range selected from 0.3 mm to 0.9 mm, 0.35 mm to 0.8 mm, 0.4 mm to 0.7 mm and 0.45 mm to 0.6 mm. Suitable and preferred embodiments were described above. As discussed, the plurality of particles used as second type may be made of the same material. In one embodiment, zirconia beads of two different sizes are used as second type. According to one embodiment, the particles of the first size are mixed with the particles of the second type in a ratio of 1:2 to 2:1, preferably 1:1.

According to one embodiment, the plurality of particles used as second type are substantially spherical and comprise or consist of zirconium, zircon (zirconium silicate), zirconia (zirconium dioxide) or yttrium-stabilized zirconium and have on average a size that lies in the range of 0.08 mm to 0.7 mm, preferably 0.09 mm to 0.6 mm. Preferably, zirconium beads are used.

According to one embodiment, the particles of the plurality of particles used as second type have a density of at least 2.0 g/cc, e.g. at least 2.5 g/cc, at least 3.0 g/cc, at least 3.5 g/cc, at least 4.0 g/cc, at least 4.5 g/cc, at least 5.0 g/cc or at least 5.5 g/cc. They may have a density that lies in a range selected from 2.0 g/cc to 15 g/cc, e.g. 2.5 g/cc to 12 g/cc, 3.0 g/cc to 10 g/cc, 3.5 g/cc to 9 g/cc, 4.0 g/cc to 8 g/cc, 4.5 g/cc to 7.5 g/cc and 5 g/cc to 7 g/cc.

Suitable amounts for the plurality of particles used as second type can be determined by the skilled person following the guidance given herein and the examples. According to one embodiment, 5 mg-500 mg of particles of the second type are used per mg of plant material.

According to one embodiment, a combination of the following disrupting particles is used for mechanically disrupting the plant sample:

(i) at least one non-spherical disrupting particle is used as first type which has the following characteristics:
it has a surface containing a first part and a second part, whereby the first part and the second part meet by forming an edge, wherein the first part is the surface of a frustum of a cone and the second part is the surface of a frustum of a cone, wherein both cones are set against each other with their larger base, the edge being formed where the larger bases meet, the larger bases being of the same diameter and wherein the at least one non-spherical particle is selected from the following group of particles that are characterized in that:
(aa) the particle comprises at least one tip which is a frustum of a cone, wherein the larger base of the frustum of the cone that provides the tip is set against the smaller base of the frustum of the cone of the second part and wherein the particle comprises a subportion that is made up of a section or a part of a ball or an ellipse which is set against the smaller base of the frustum of the cone of the first part, wherein preferably, the subportion that is made up of a section or a part of a ball or an ellipse is a semi-sphere,
(bb) the particle comprises at least two tips, wherein both tips are a frustum of a cone, wherein the larger base of the frustum of a cone is set against the smaller base of the frustum of the cone of the first part and the larger base of the frustum of a cone is set against the smaller base of the frustum of the cone of the second part,
(cc) the particle comprises two subportions, wherein each subportion is made up of a section or a part of a ball or an ellipse, wherein the first subportion that is made up of a section or a part of a ball or an ellipse is set against the smaller base of the frustum of the cone of the first part and the second subportion that is made up of a section or a part of a ball or an ellipse is set against the smaller base of the frustum of the cone of the second part, and/or
(dd) the particle comprises two semi-spheres wherein the first semi-sphere is set against the smaller base of the frustum of the cone of the first part and the second semi-sphere is set against the smaller base of the frustum of the cone of the second part;
it has a weight of at least 300 mg, preferably at least 400 mg, more preferably at least 500 mg; and
it has a size of at least 1.5 mm, preferably at least 2 mm, more preferably at least 3 mm;
   and
   (ii) the second type is provided by a plurality of substantially spherical zirconia beads, preferably having a size that lies in the range of 0.08 mm to 0.7 mm, more preferably 0.09 mm to 0.6 mm. The at least one non-spherical disrupting particle used as first type is preferably made of steel, stainless steel, tungsten or other heavy metals as discussed above. As disclosed herein, the plurality of particles used as second type may have at least two different sub-sizes that lie within this broader range. Details have been described above.

According to one embodiment, no further type of particles is used in addition to the first and second type of disrupting particles.

Mechanical Disruption with the First and Second Type of Particles

Disruption of the plant sample and the microorganisms comprised in the plant sample with the first and second type of disrupting particles may be performed sequentially or simultaneously. Preferably, it is performed simultaneously.

The disrupting particles of the first and second type may be comprised in a container, which preferably also comprises the lysis solution. In one embodiment, the lysis solution and the disrupting particles of the first and/or second type are comprised in the same compartment of the container and are provided in form of a composition. The plant sample from which the nucleic acids are to be isolated can be added to the container. The container is then closed and mechanical disruption can be initiated. If the first and second type are not provided in the same container, the missing type can be added subsequently to allow simultaneous or sequential disruption with the two types of particles.

The container for receiving the tissue material may be any suitable container or reaction vessel, which is preferably inert with respect to the agents used in the disruption treatment, which exhibits enough mechanical stability to withstand the forces of the disrupting particles without being destroyed or abraded, which exhibits a suitable size for receiving the plant sample material, the lysis solution and the one or more selected disrupting particle and still provides suitable space to allow agitation and movement of the inserted components to effect disruption and thus lysis of the plant material, and which can suitably be used with the device which is used for effecting the milling or grinding of the tissue material by the disrupting particles. Suitable container or reaction vessels (tubes) are known and commonly available.

According to one embodiment, the plant sample is homogenized to provide a lysate. As is demonstrated by the examples, the present technology efficiently homogenizes a wide range of plant samples.

Mechanical disruption with the disrupting particles of the first and/or second type may include the use of bead beating and/or homogenizing devices. Suitable devices may include but are not limited to high-performance mixer or high-speed mixer, as well as low-power mixers, such as common laboratory vortexer, bench-top vortexer, or common lab shaker (e.g. horizontal shaker). Disruption may be performed using a vortex mixer with bead tube adapter or beating devices, such as TissueLyzer II (QIAGEN), AMBION™ Vortex Adapter (Thermo Fisher Scientific, Waltham, Mass.) and the Omini Bead Rupter Homogenizer, OMNI Intl Kennesaw, Ga.), and various homogenizers by OPS Diagnostics. High-power or high-performance mixer usually work with a frequency of 15 to 60 Hz. Low-power mixer such as in particular common vortexer usually work with a force of 150 up to 3200 rpm. Applying a reduced mechanical power, e.g. from a low-power mixer or vortexer can be advantageous for preserving the quality of the released nucleic acids and avoid damages or deterioration of the nucleic acids. In one embodiment, a high-speed shaker (e.g. 15-60 Hz) is used. In embodiments, it achieves oscillations/minute that are in a range of 150-2500, e.g. 180-1800. Suitable and advantageous duration for mechanical disruption can be determined by the skilled person. For example, one disruption cycle may comprise mechanical disruption with the one or more disrupting particles for 30 sec-20 min, 1 min to 15 min, 1.5 min to 10 min and 2 min to 7 min. Two or more disruption cycles can be performed if desired to achieve a good homogenization of the lysate.

### Preferred Lysis Conditions

The liquid lysis composition preferably comprises at least one chaotropic agent. According to one embodiment, the liquid lysis composition is a solution, preferably an aqueous solution. The solid disrupting particles can be comprised in the solution.

According to one embodiment, the chaotropic agent is a chaotropic salt.

According to one embodiment, the chaotropic agent is selected from sodium thiocyanate, sodium carbonate, potassium thiocyanate, ammonium thiocyanate, lithium thiocyanate, lithium perchlorate, guanidine sulfate, and combinations thereof. Such chaotropic agents can be used to generate a lysate.

According to one embodiment, the chaotropic agent is selected from sodium thiocyanate, potassium thiocyanate, ammonium thiocyanate, lithium thiocyanate and combinations thereof. Such chaotropic agents are particularly suitable to generate a lysate.

According to one embodiment, the chaotropic agent is NaSCN. According to one embodiment, the lysis composition comprises only one chaotropic agent and preferably comprises NaSCN as only chaotropic agent.

NaSCN, Na₂CO₃, KSCN, NH₄SCN, LiSCN, LiClO₄, guanidine sulfate are relatively mild chaotropic agents which is advantageous for the present method which combines in embodiments such mild lysis with mechanical disruption using the first and second type of disrupting particles. Preferably, the relatively mild chaotropic agent is NaSCN.

The relatively mild chaotropic agents which may be used as chaotropic agent in the liquid lysis composition may include salts having the strong anion, SCN⁻, paired with a cation weaker than Mg²⁺ in solubilizing proteins; salts having the strong anion, ClO₄⁻, paired with a cation weaker than Mg²⁺ in solubilizing proteins; and salts having the weak anion, CO₃²⁻, paired with a cation stronger than NH₄⁺ in solubilizing proteins.

The relatively mild chaotropic agents (e.g., NaSCN) strike a desirable balance between a stronger chaotropic agent such as GuSCN or GuCl and a weaker chaotropic agent such as RbSCN. The less aggressive chaotropic agent can effectively solubilize biomolecules during disruption with the disrupting particle to make them available for downstream isolation. Strong chaotropic agents and detergents (e.g., SDS), on the other hand, can achieve complete cell lysis but at the expense of degraded biomolecules (e.g., degraded nucleic acids). The less aggressive chaotropic agents that are preferably used in conjunction with the present method are unique in their capacity to solubilize biomolecules (e.g., nucleic acids) while minimizing degradation of nucleic acids. Therefore, combining such mild chaotropic agent with the mechanical sample disruption process using two types of disrupting particles as taught herein is particularly advantageous and provides an improvement over prior art methods.

The concentration of the at least one chaotropic agent in the liquid lysis composition and/or the lysis mixture (comprising the plant sample) may be 2.5M or less, e.g. 2M or less, 1.75M or less, 1.5M or less, 1.3M or less, 1.2M or less or 1.125M or less. Suitable concentrations of the at least one chaotropic agent in the liquid lysis composition which preferably is a lysis solution and/or the lysis mixture may be in the range selected from 0.5 to 2.5M, e.g. 0.6M to 2M, 0.7M to 1.75M, 0.75M to 1.5M and preferably 0.8 to 1.25M. If multiple chaotropic agents are present in the liquid lysis composition, which preferably is a lysis solution, the total concentration of chaotropic agents in the liquid lysis composition, respectively lysis solution may be and preferably lies in the above described range. The same applies with respect to the lysis mixture. The solid disrupting particles are not considered for determining the concentration.

The chaotropic agent is preferably a thiocyanate salt as described above, more preferably NaSCN. The above concentrations were found particularly suitable for such mild thiocyanate salts, such as NaSCN. Particularly preferred is a concentration of NaSCN in the liquid lysis composition and/or in the lysis mixture in the range of 0.7M to 1.75M, e.g. 0.75M to 1.5M and preferably 0.8 to 1.25M.

According to one embodiment, the method further comprises adding at least one phosphate. The at least one phosphate is added prior to contacting the lysed sample with at least one inhibitor removing agent, if such inhibitor removing agent is used (see below). Without wishing to be bound by theory, it is believed that the free phosphate group (PO₄³⁻) prevents or reduces complex formation between the subsequently used inhibitor removing agent (e.g., AlCl₃) and the phosphodiester groups of nucleic acids by competitively interacting with the inhibitor removing agent.

Preferably, the at least one phosphate is present during mechanical disruption. Preferably, the at least one phosphate is included in the liquid lysis composition which is, as described, preferably a lysis solution. Therefore, the liquid lysis composition comprises in an advantageous embodiment the at least one chaotropic agent and the at least one phosphate. According to one embodiment, the liquid lysis composition comprises sodium thiocyanate and a phosphate.

Exemplary phosphates include phosphate monobasics, phosphate dibasics, and phosphate tribasics, and other compounds that contain one or more free phosphate groups, such as sodium phosphate monobasic, sodium phosphate dibasic, sodium phosphate, potassium phosphate monobasic, potassium phosphate dibasic, potassium phosphate, ammonium phosphate monobasic, ammonium phosphate dibasic, ammonium phosphate, lithium phosphate monobasic, lithium phosphate dibasic, lithium phosphate, trisodium phosphate, sodium poly(vinylphosphonate), sodium hexametaphosphate, pyrophosphate, sodium triphosphate, sodium polyphosphate, other phosphorus-containing oxyanions, and combinations thereof. The cationic moieties in the phosphates include but are not limited to ammonium, sodium, potassium, and lithium. In one embodiment, the cationic moiety is provided by an alkali metal ion, preferably selected from sodium, potassium and lithium, more preferably sodium. Preferably, the phosphate is a phosphate dibasic and more preferably is sodium phosphate dibasic.

The concentration of the at least one phosphate in the liquid lysis composition, the lysis mixture (comprising the plant sample) and/or the lysed sample may be selected from 0.05 to 0.75M, 0.06M to 0.6M, 0.075M to 0.5M, 0.1M to 0.3M and 0.1M to 0.25M or may be 0.125M to 0.2M. As disclosed herein, it is preferred to comprise the at least one phosphate in the liquid lysis composition. The concentration of the at least one phosphate in the liquid lysis composition, which preferably is a lysis solution, is preferably in the range of 0.1 M to 0.3M or 0.1M to 0.2M.

According to one embodiment, the liquid lysis composition comprises sodium thiocyanate and at least one phosphate, preferably sodium phosphate dibasic.

According to one embodiment, the liquid lysis composition and/or the liquid lysis mixture comprises sodium thiocyanate in a concentration selected from 0.7M to 1.75M, 0.75M to 1.5M and preferably 0.8 to 1.25M and the at least one phosphate, preferably sodium phosphate dibasic, in a concentration selected from 0.075M to 0.3M, 0.1 to 0.25M and 0.1 M to 0.2M. Preferably, the concentration of the at least one phosphate, preferably sodium phosphate dibasic, is in the range of 0.1M to 0.3M.

According to one embodiment, the liquid lysis composition and/or the liquid lysis mixture comprises sodium thiocyanate in a concentration of 0.7M to 1.75M and the at least one phosphate, preferably sodium phosphate dibasic, in a concentration of 0.075M to 0.3M.

According to one embodiment, the liquid lysis composition and/or the liquid lysis mixture comprises sodium thiocyanate in a concentration of 0.75M to 1.5M and the at least one phosphate, preferably sodium phosphate dibasic, in a concentration of 0.1 to 0.3M.

According to one embodiment, the liquid lysis composition and/or the liquid lysis mixture comprises sodium thiocyanate in a concentration of 0.8 to 1.25M and the at least one phosphate, preferably sodium phosphate dibasic, in a concentration of 0.1 to 0.25M.

A lysis composition such as a lysis reagent can be combined with the disrupting particles before the plant sample is added. However, the plant sample may also be contacted with the lysis reagent before adding the first and/or second type of disrupting particles.

The lysis reagent, which preferably is a lysis solution, may comprise

(i) one or more chaotropic agents selected from sodium thiocyanate, sodium carbonate, ammonium thiocyanate, potassium thiocyanate, lithium thiocyanate, lithium perchlorate, guanidine sulfate, and combinations thereof, and
(ii) one or more phosphates.

Details of the chaotropic agent and the at least one phosphate have been described above and it is referred to the respective disclosure. The concentrations described above for the liquid lysis composition also apply to the lysis reagent, which preferably is a lysis solution. Hence, according to one embodiment, the concentration of the at least one chaotropic agent in the lysis reagent may be 2.5M or less, e.g. 2M or less, 1.75M or less, 1.5M or less, 1.3M or less, 1.2M or less or 1.125M or less. Suitable concentrations of the at least one chaotropic agent in the lysis reagent may be in the range selected from 0.5 to 2.5M, e.g. 0.6M to 2M, 0.7M to 1.75M, 0.75M to 1.5M and preferably 0.8 to 1.25M. If multiple chaotropic agents are present in the lysis reagent, the total concentration of chaotropic agents in the lysis reagent may be and preferably lies in the above described range. The chaotropic agent is preferably a thiocyanate salt as described above, more preferably NaSCN. Particularly preferred is a concentration of NaSCN in the lysis reagent in the range of 0.7M to 1.75M, e.g. 0.75M to 1.5M and preferably 0.8 to 1.25M. The concentration of the at least one phosphate in the lysis reagent may be selected from 0.05 to 0.75M, e.g. 0.06M to 0.6M, 0.075M to 0.5M, 0.1M to 0.3M and 0.1 M to 0.25M. It is referred to the above disclosure.

According to one embodiment, the lysis reagent comprises sodium thiocyanate in a concentration selected from 0.7M to 1.75M, 0.75M to 1.5M and preferably 0.8 to 1.25M and the at least one phosphate, preferably sodium phosphate dibasic, in a concentration selected from 0.075M to 0.3M, 0.1 to 0.25M and 0.1M to 0.2M, or 0.125M to 0.2M. Preferably, the concentration of the at least one phosphate, preferably sodium phosphate dibasic, is in the range of 0.1M to 0.3M. It is referred to the above disclosure.

Preferably, the lysis reagent comprises sodium phosphate dibasic and sodium thiocyanate.

The one or more solid disrupting particles may be comprised in the lysis reagent. As described herein, the lysis reagent may be comprised in a container which additionally comprises the first and/or second type of solid disrupting particles. The solid disrupting particles may be contained in, e.g. immersed in the lysis reagent. This embodiment is advantageous, because the plant sample may be added to the liquid lysis composition which comprises the at least one chaotropic agent and the first and/or second type of disrupting particles and mechanical disruption can be initiated.

In certain other embodiments, the liquid lysis composition does not include any detergent, such as SDS.

A liquid lysis composition, which may be a lysis solution, may optionally further contain one or more buffer substances.

The pH of the liquid lysis composition may be at least 3, e.g. at least 4 or at least 5. E.g., the pH of the liquid lysis composition may be in the range of pH 3 to pH 10, e.g. pH 4 to pH 9 and pH 5 to 8.0.

The liquid lysis composition, which preferably is a lysis solution, may comprise, consist essentially of, or consist of one or more chaotropic agents and one or more phosphates, both as described above may be an aqueous solution. For mechanical disruption, the solid disrupting particles as described are included in the liquid lysis composition, which may be a lysis solution. Preferably, the one or more relatively mild chaotropic agents comprise or is NaSCN. The one or more phosphates preferably comprise or are sodium phosphate dibasic.

An exemplary preferred lysis solution comprises, consists essentially of, or consists of 0.5 to 2M NaSCN and 0.1 to 0.2M Na₂HPO₄.

### Preferred Further Steps for Processing the Lysate

Further optional but preferred steps for processing the lysate are described in the following:

### Clearing the Lysate

The method may furthermore comprise clearing the lysate. As discussed herein, disrupting the plant sample provides a lysed mixture which may comprise solid components from the plant sample and a liquid fraction which comprises released nucleic acids. As disclosed herein, the mechanical disruption which is supported by the used lysis chemistry advantageously allows to efficiently homogenizing different types of plant samples. It is preferred to separate the solid components from the liquid fraction and to further process the liquid fraction as lysed sample.

The lysate clearing step may comprise separating the lysed mixture that is obtained upon disrupting the plant sample into a solid fraction and a liquid fraction. The liquid fraction comprises the nucleic acids (and may still comprise some plant particles) and the liquid fraction may be further processed as lysed sample. The solid components may be discarded. Separation of the liquid fraction may be assisted by sedimentation, centrifugation, or filtration, preferably by centrifugation. Also combinations of such methods can be used. The separated liquid fraction (e.g. supernatant) may then further processed as lysed sample.

Contacting the Lysed Sample with at Least One Precipitating Agent and at Least One Inhibitor Removing Agent and Providing a Mixture

The method may further comprise contacting the lysed sample, which is optionally cleared, with at least one protein precipitating agent and at least one inhibitor removing agent and providing a mixture.

The lysed sample may be contacted with at least one protein precipitating agent and at least one inhibitor removing agent and a mixture is provided. This step may comprise agitating the mixture, e.g. by vortexing.

### Protein Precipitating Agent

According to one embodiment, the at least one protein precipitating agent is selected from ammonium acetate, ammonium sulfate, potassium acetate, sodium acetate, sodium chloride and cesium acetate.

Some of the precipitating agents (e.g., ammonium acetate) may function as a protein precipitating agent at a relatively high concentration (e.g., at 1 to 2 M in the mixture comprising the lysed sample, the precipitating agent, and one or more inhibitor removing agents as described below) but as a molecular screen at a relatively low concentration (e.g., at a concentration 5 to 15 times less than the concentration when functioning as a protein precipitation agent). The use of ammonium acetate is preferred.

According to one embodiment, the concentration of the at least one precipitating agent in the mixture is in a range selected from 0.1 to 4M, e.g. 0.2M to 3M, 0.3M to 2.5M, 0.4M to 2.25M, 0.5M to 2M and 0.6M to 1.75M. According to one embodiment, ammonium acetate is used in such concentration range, preferably is present in the mixture of step (b) in a concentration that lies in the range of 0.5M to 2M or 0.6M to 1.75M.

### Inhibitor Removing Agent

Exemplary inhibitor removing agents include aluminum ammonium sulfate, aluminum ammonium sulfate dodecahydrate, ammonium sulfate, aluminum potassium sulfate, aluminum chlorohydrate, calcium oxide, iron (III) chloride, iron (II) sulfate, sodium aluminate, sodium silicate, magnesium chloride, aluminum chloride, aluminum sulfate, erbium (III) acetate, erbium (III) chloride, holmium chloride, zirconium (IV) chloride, hafnium (IV) chloride, and combinations thereof.

According to one embodiment, the inhibitor removing agent comprises a trivalent cation. Preferably, the inhibitor removing agents include aluminum chloride, aluminum sulfate, erbium (III) acetate, erbium (III) chloride, holmium chloride, zirconium (IV) chloride, hafnium (IV) chloride, and combinations thereof.

Thus, according to one embodiment, the at least one inhibitor removing agent is selected from aluminum chloride, erbium (III) acetate, erbium (III) chloride, holmium chloride, hafnium (IV) chloride, zirconium (IV) chloride, guanidine sulfate, and combinations thereof and wherein preferably, the inhibitor removing agent is aluminum chloride.

The use of a trivalent aluminum salt such as aluminum chloride is particularly preferred. The use of aluminum chloride is advantageous because it can be used over a broad pH range.

As discussed herein, at least one phosphate may be added, preferably during sample lysis. It serves the purpose to prevent the precipitation of nucleic acids such as in particular DNA from the mixture to prevent a loss of nucleic acid material.

The pH in the mixture may be at least 3, e.g. at least 4 or at least 5. E.g., the pH during step (b) may be in the range of pH 3 to pH 10, e.g. pH 4 to pH 9 and pH 5 to 8.0.

According to one embodiment, the concentration of the at least one inhibitor removing agent in the mixture is in the range selected from 1 to 150 mM, e.g. 5 mM to 125 mM, 10 mM to 100 mM, 15 mM to 75 mM and 20 mM to 65 mM. As discussed above, the use of a trivalent aluminum salt such as aluminum chloride is particularly preferred and it is in one embodiment used in such concentration. Particularly preferred is a concentration of aluminium chloride that is selected 15 mM to 75 mM, e.g. 20 mM to 65 mM or 25 mM to 55 mM.

The lysed sample comprises a contaminant or inhibitor that forms a complex with the one or more inhibitor removing agents, and the complex is precipitated and removed by the one or more inhibitor removing agents. As described herein, plant samples often comprise a large amount of inhibitors, including e.g. polysaccharides and polyphenolic compounds. Such inhibitors remain present in the lysed sample. The present method allows to efficiently remove inhibitors, thereby allowing to isolate high quality nucleic acids, such as DNA.

According to one embodiment, the precipitating agent is ammonium acetate, and the inhibitor removing agent is aluminum chloride.

The protein precipitation step and the inhibitor removing step can be performed sequentially. However, preferably, they are performed simultaneously.

According to one embodiment, which is preferred, the lysed sample is contacted with a composition comprising the at least one precipitating agent and the at least one inhibitor removing agent. As disclosed, the lysed sample is preferred a cleared lysate.

The one or more precipitating agents and the one or more inhibitor removing agents may be added in form a composition either in solid form or as a solution, preferably as a solution. Preferably, the composition is an aqueous solution. It can be added to the lysed sample.

According to one embodiment, the composition comprises, consists essentially of, or consists of
(i) one or more precipitating agents selected from ammonium acetate, ammonium sulfate, potassium acetate, sodium acetate, sodium chloride, cesium acetate, and combinations thereof,
(ii) one or more inhibitor removing agents selected from aluminum chloride, erbium (III) acetate, erbium (III) chloride, holmium chloride, hafnium (IV) chloride, zirconium (IV) chloride, and combinations thereof, and
(iii) optionally water.

In the embodiments where the composition is a solution the total concentration of the one or more precipitating agents in the solution that is added is in the range of 0.5 M to 10M, 1 to 8M, or 1.5 to 7.5M, preferably 1M to 6M, 1.5M to 5.5M, 2M to 5M, 2.5 to 4.5M and 3M to 4M. This is particularly suitable when the precipitating agent functions as a protein precipitating agent. The precipitating agent can be ammonium acetate and the described concentrations are particularly suitable when using ammonium acetate. The composition can be added to the lysed sample.

In the embodiments where the composition is a solution the total concentration of the one or more inhibitor removing agents in the solution that is added is in the range of 10 to 500 mM, e.g. 25 mM to 400 mM, 50 mM to 350 mM, 75 mM to 300 mM, 90 mM to 250 mM, preferably 50 mM or 100 mM to 200 mM, such as 50 mM to 175 mM or 75 mM to 150 mM. As discussed above, the use of a trivalent aluminum salt such as aluminum chloride is particularly preferred as inhibitor removing agent and it is in one embodiment comprised in such concentration in the solution. According to one embodiment, the solution that is added comprises aluminum chloride in a concentration of 50 mM to 250 mM. Particularly preferred concentrations of aluminum chloride include 50 mM to 200 mM, 50 mM to 175 mM and 75 mM to 150 mM.

Exemplary preferred solutions that comprise a precipitating agent and an inhibitor removal agent include:
(1) a solution containing 1 to 8M (preferably 2.5 to 5M) ammonium acetate and 20 to 200 mM aluminum chloride;
(2) a solution containing 1 to 10M (preferably 1 to 8M) sodium acetate and 20 to 200 mM aluminum chloride;
(3) a solution containing 1 to 8M (preferably 1 to 5M) cesium acetate and 20 to 200 mM aluminum chloride;
(4) a solution containing 1 to 8M (preferably 2.5 to 5M) ammonium acetate and 20 to 200 mM erbium (III) acetate;
(5) a solution containing 1 to 10M (preferably 1 to 8M) sodium acetate and 20 to 200 mM erbium (III) acetate;
(6) a solution containing 1 to 8M (preferably 1 to 5M) cesium acetate and 20 to 200 mM erbium (III) acetate;
(7) a solution containing 1 to 8M (preferably 2.5 to 5M) ammonium acetate and 20 to 200 mM erbium (III) chloride;
(8) a solution containing 1 to 10M (preferably 1 to 8M) sodium acetate and 20 to 200 mM erbium (III) chloride;
(9) a solution containing 1 to 8M (preferably 1 to 5M) cesium acetate and 20 to 200 mM erbium (III) chloride;
(10) a solution containing 1 to 8M (preferably 2.5 to 5M) ammonium acetate and 20 to 200 mM holmium chloride;
(11) a solution containing 1 to 10M (preferably 1 to 8M) sodium acetate and 20 to 200 mM holmium chloride; and
(12) a solution containing 1 to 8M (preferably 1 to 5M) cesium acetate and 20 to 200 mM holmium chloride.

According to one embodiment, the precipitating agent in the composition that is added to the lysed sample is selected from ammonium acetate, sodium acetate, cesium acetate, or a combination thereof, preferably ammonium acetate and the inhibitor removing agent is aluminum chloride.

According to one embodiment, no precipitation, centrifugation or filtration has been performed between contacting the lysed sample with the at least one precipitating agent and contacting the lysed sample with the at least one inhibitor removing agents. As disclosed herein, it is preferred to add the precipitating agent and the inhibitor removal agent at the same time, e.g. by adding a liquid composition that comprises the at least one precipitating agent and the at least one inhibitor removing agent.

As used herein, the term “inhibitor” in particular refers to any substance that interferes with a reaction involving DNA and/or RNA isolated from a sample, and has a detrimental effect on DNA and/or RNA manipulation. Inhibitors include, for example, inhibitors of an enzymatic reaction that uses DNA or RNA as a substrate and a contaminant that disrupts hybridization of DNA or RNA. Inhibitors may include humic substances. They comprise polycyclic aromatics to which saccharides, peptides, and phenols are attached. Additional exemplary inhibitors include decomposing plant materials, organic compounds from compost, phenolics, phenolic polymers or oligomers, polyphenol, polysaccharides, and tannin. Examples of polysaccharide inhibitors include but are not limited to pectin and xylan. As disclosed herein, the present method improves sample lysis thereby advantageously increasing the release of nucleic acids, such as in particular DNA, into the lysate. This improved lysis may furthermore release more inhibitors into the lysate and hence the DNA containing supernatant that can be obtained from the lysate as described herein. Therefore, it is advantageous and important to efficiently remove inhibitors in order to provide high quality nucleic acids.

The inhibitor removing agent is capable of substantially removing one or more inhibitors from the lysed sample. After obtaining a liquid phase from the mixture (see below), an inhibitor is substantially removed. E.g. 20% or less, preferably 18% or less, 15% or less, 13% or less, or 10% or less, more preferably 5% or less, 3% or less, 2% or less, or 1% or less of the inhibitor from the sample remains in the liquid phase after separating the mixture into a solid phase and a liquid phase.

Obtaining a Liquid Phase from the Mixture

The method may further comprise obtaining a liquid phase from the mixture.

During or subsequent to the protein precipitation and inhibitor removal, solid components are generated, e.g. by precipitation and complexing processes. It is therefore preferred to perform a step comprising obtaining a liquid phase from the mixture. This can be assisted again by sedimentation, filtration or preferably centrifugation. Also a combination of according techniques can be used.

According to one embodiment, this step accordingly comprises removing solid components comprised in the provided mixture to obtain a liquid phase that comprises the nucleic acids. The liquid phase can be provided, respectively obtained in form of a supernatant.

The mixture may be centrifuged, filtrated, precipitated, or otherwise treated to separate its solid phase from its liquid phase wherein the one or more inhibitor removing agents are primarily (more than 50%) in the solid phase. The solid phase may be provided in form of a pellet. The one or more inhibitor removing agents form complexes with inhibitors and other contaminating materials from the sample, which complexes are precipitated out or otherwise removed from the liquid phase in said step.

In certain embodiments, more than 60%, 70%, or 80%, preferably more than 90%, or more preferably more than 95% of the one or more inhibitor removing agents are removed from the liquid phase in this step.

Optionally Isolating Nucleic Acids, Preferably DNA, from the Liquid Phase

The liquid phase obtained may be subsequently used for isolating nucleic acids therefrom.

The term “nucleic acids” as used herein include single- or double-stranded nucleic acids and can be selected from DNA and RNA. Any methods suitable for isolating DNA, RNA, or both from a solution may be used. Suitable methods are well known to the skilled person and therefore, do not need to be described in detail. Preferably, the nucleic acid isolated from the liquid phase is DNA.

The improved lysis and inhibitor removal that is achieved with this embodiment, provides a liquid phase that comprises large amounts of nucleic acids, including microbial nucleic acids (due to the improved lysis) and which is advantageously depleted from inhibitors (due to the use of the precipitating agent and inhibitor removing agent). Therefore, nucleic acids such as DNA can be isolated with high yield and purity from the provided liquid phase. Essentially any nucleic acid isolation method can be used in order to isolate the nucleic acids, preferably DNA, from the provided liquid phase. Exemplary methods are described in conjunction with the method according to the second aspect to which it is referred.

As will be appreciated from the present disclosure, the method does not require the use of phenol and/or CTAB. Therefore, in embodiments, the method does not involve the use of phenol and/or CTAB. In embodiments, no detergent is added to assist the lysis. In embodiments, the method does not involve the use of proteolytic enzymes such proteinase K to assist the lysis.

### The Method According to the Second Aspect

According to a second aspect a method for isolating nucleic acids including microbial nucleic acids from a plant sample is provided, comprising

(a) performing the lysis method according to the first aspect;
(b) isolating nucleic acids from the lysed and optionally further processed sample; and
(c) optionally sequencing isolated nucleic acid, preferably sequencing isolated DNA.

The details and preferred embodiments of the lysis method that is performed in step (a) were described above and it is referred to the respective disclosure which also applies here.

In addition, suitable and preferred embodiments for further processing the lysed sample, were described above and it is referred to the respective disclosure. Preferably, step (a) comprises
contacting the lysed sample, which is optionally cleared, with at least one protein precipitating agent and at least one inhibitor removing agent and providing a mixture; and
obtaining a liquid phase from the mixture.

Nucleic acids, such as preferably DNA, can be isolated from the liquid phase. It is referred to the above disclosure.

The term “nucleic acids” as used herein include single- or double-stranded nucleic acids and can be selected from DNA and RNA. Any methods suitable for isolating DNA, RNA, or both from a solution may be used. Suitable methods are well known to the skilled person and therefore, do not need to be described in detail. Preferably, the isolated nucleic acid is DNA.

Nucleic acids such as DNA can be isolated with high yield and purity from the provided liquid phase. Essentially any nucleic acid isolation method can be used in step (b) in order to isolate the nucleic acids, preferably DNA.

Preferably, a nucleic acid-binding solid support is used in nucleic acid isolation. Exemplary solid support includes silica matrices, glass particles, diatomaceous earth, magnetic beads, nitrocellulose, nylon, and anion-exchange materials. The solid support may be in the form of loose particles, filters, membranes, fibers or fabrics, or lattices, and contained in a vessel, including tubes, columns, and preferably a spin column.

To facilitate or strengthen binding of nucleic acids to a solid support, a binding solution may be used. The binding solution may be added during sample lysis (e.g., after mechanical disruption of the sample in the presence of a lytic reagent) before contacting the sample material with a protein precipitating agent and an inhibitor removing agent during the inhibitor removal process. Alternatively, the binding solution may be added to the liquid phase obtained after the inhibitor removal process.

Exemplary DNA binding solution may comprise a chaotropic agent (e.g., GuSCN or GuHCl), an alcohol (e.g., ethanol or isopropanol), or both. It may further comprise a buffer substance, such as Tris HCl.

In the embodiments where both DNA and RNA are isolated from a sample, DNA isolation and RNA isolation may be performed in parallel. In other words, the liquid phase of step (b) is divided into at least two portions: one for DNA isolation, and one for RNA isolation. DNA and RNA may also be isolated sequentially. When aiming at isolating RNA, an RNase inhibitor may be used in lysis step (a) to protect the released RNA.

Methods for sequentially isolating DNA and RNA are known (see e.g., U.S. Pat. No. 8,889,393, WO 2004/108925). Preferably, a solid support for binding DNA and a solid support for binding RNA are used. The solid support for binding DNA may be identical to or different from the solid support for binding RNA. When an identical solid support is used for DNA and RNA isolation, differential binding of DNA and RNA to the solid support may be achieved by adjusting the component(s) and/or their concentration(s) of binding mixtures. For example, a silica spin column may be used to bind DNA first while the flow through may be mixed with ethanol, and the resulting mixture is applied to a second silica spin column to bind RNA (Triant and Whitehead, Journal of Heredity 100:246-50, 2009).

After binding to a solid phase, DNA or RNA bound to the solid phase may be washed, and subsequently eluted from the solid phase. DNA wash solution may comprise a chaotropic agent (e.g., GuHCl), an alcohol (e.g., ethanol, isopropanol), or both. It may further comprise a buffer substance (e.g., Tris HCl), a chelating agent (e.g., EDTA (ethylenediaminetetraacetic acid)), and/or a salt (e.g., NaCl). DNA elution solution may be a buffer (e.g., a Tris buffer) or water.

RNA binding solution may comprise alcohol (e.g., ethanol, isopropanol) and optionally another organic solvent (e.g., acetone). RNA wash solution may comprise one or more of the following: a buffer substance (e.g., Tris HCl and Tris base), a chelating agent (e.g., EDTA), an alcohol, and a salt (e.g., NaCl). RNA may be eluted from a solid support using DEPC-treated or other RNase-free water.

According to one embodiment, at least DNA is isolated. According to one embodiment, DNA is isolated while depleting RNA during the performance of the method. Furthermore, RNA may be destroyed by using RNase.

The method may further comprise analyzing the isolated nucleic acids in step. Such analysis steps may comprise any common analysis technique such as PCR, qPCR, RT-PCR, or nucleic acid sequencing. The DNA provided by the present method is in particular suitable for sequencing applications such as next generation sequencing. E.g. sequencing can be performed to identify the plant microbiome. The plant microbiome can be e.g. determined by sequencing in order to look at commensal and/or pathogenic species.

### Plant Samples

The present method is particularly suitable for processing various plant sample types. Advantageously, the present method can be used for different types of plant samples while ensuring good results with respect to yield and purity.

The term “plant” in particular refers to whole plants, plant organs, plant tissues, roots, seeds, plant cells, and progeny of the same. Plant samples include, without limitation, seeds, embryos, meristematic regions, callus tissue, leafs, roots, shoots, gametophytes, sporophytes, pollen and microspores. Plant parts include differentiated and undifferentiated tissues including, but not limited to roots, stems, shoots, leaves, pollens, seeds, tumor tissue and various forms of cells, as well as fruit and flowers.

As mentioned above, the term “plant” refers also to a part of a plant like leaf (blade (base, midrib, veins, margin, apex) and petiole (leafstalk) stipule), stem, root (primary root, lateral root, root hairs, root tip, root cap) needle, flowers (sepal, filament, anther, pollen, petal, stigma, style, ovary, ovule) fruits, buds (axillary bud, apical bud/terminal bud) nodes, internodes. For example leafs include any types of leafs like A) simple, pinnately veined leaves (Oak, Birch); B) Simple, palmately veined leafs (Sweet gum); C) Pinnately compound veined (Walnut); D) Palmately compound leaf (Buckeye); E) Parallel veins (Grass); F) Opposite leaves (Maple) G) Alternating leafs (Elm) H) Needle leafs: Spruces (4 sided needles—Sitka spruce), Pines (2, 3 or 5 needle bundles—Ponderosa pine), Firs (flat needles—Hemlock), Scale leaves (Sequoia redwood).

According to one embodiment, the plant sample from which nucleic acids are isolated is selected from leaf, needle, root, stem and seed. Furthermore, the plant sample may be selected from fruit and flower. According to one embodiment, the plant sample is obtained from a plant selected from agricultural crop, such as wheat, rice, apple, coffee, tobacco, corn, sunflower, grass and so on. A further common plant sample is cotton.

Exemplary common samples from which nucleic acids such as in particular DNA can be isolated include but are not limited to leaf tissues, such as soft or fibrous leaf tissues, e.g. grape leaf, strawberry leaf, cotton leaf, grass leaf, rice leaf and/or mint leaf, stems, such as e.g. tomato stem, needles, such as pine needle and seeds.

If the plant sample comprises a large amount of phenolic compounds, it is within the scope of the method to add a further compound during lysis that removes phenolic compounds. A suitable example is PVP. This can be advantageous for samples such as pine needle or strawberry leaf.

The plant sample comprises or is suspected of comprising microorganisms. Microorganisms comprised in plant samples may be selected from bacteria and fungi, such as gram-positive bacteria, gram-negative bacteria, fungus, mold and spores, or a combination of the foregoing. In one embodiment, the microorganisms are bacteria. In a plant sample that comprises microorganisms, the microorganisms may be present on, around or within the plant sample. Microorganisms such as bacteria may optionally be comprised in root samples, on leaf surfaces and/or lesions or tumors in the plant tissue. The method of the invention which uses a combination first and second type of disrupting particles is particularly effective in disrupting various plant samples, thereby also releasing microorganisms comprised within the plant sample and thereby rendering them accessible for efficient lysis. Suitable plant samples were described above.

### The System According to the Third Aspect

According to a third aspect, a lysis system, preferably a kit, is provided for releasing microbial nucleic acids from microorganisms comprised in a plant sample, comprising

(a) a liquid lysis composition
(b) at least two types of solid disrupting particles, wherein
(i) the first type is provided by one or more disrupting particles having a size of at least 1.5 mm; and
(ii) the second type is provided by a plurality of disrupting particles having a size of less than 1 mm.

Details for the first and second type of solid disrupting particles and preferred combinations were described above in conjunction with the method according to the first aspect and it is referred to the respective disclosure which also applies here. Details of the liquid lysis composition were also described in conjunction with the first aspect and it is referred thereto.

The first and second type of disrupting particles may be comprised either in separate containers or in the same container, and preferably are comprised in the same container.

According to one embodiment, the liquid lysis composition comprises at least one chaotropic agent, preferably selected from sodium thiocyanate, sodium carbonate, ammonium thiocyanate, potassium thiocyanate, lithium thiocyanate, lithium perchlorate, guanidine sulfate, and combinations thereof. The chaotropic agent is preferably a chaotropic salt. It may be selected from sodium thiocyanate, potassium thiocyanate, ammonium thiocyanate, lithium thiocyanate and combinations thereof. The chaotropic agent is preferably NaSCN.

According to one embodiment, the lysis composition has one or more of the following characteristics:

(i) the concentration of the at least one chaotropic agent in the liquid lysis composition is selected from 2.5M or less, e.g. 2M or less, 1.75M or less, 1.5M or less, 1.3M or less, 1.2M or less and 1.125M or less;
(ii) the concentration of the at least one chaotropic agent in the liquid lysis composition lies in a range selected from 0.5 to 2.5M, e.g. 0.6M to 2M, 0.7M to 1.75M, 0.75M to 1.5M and preferably 0.8 to 1.25M; and/or
(iii) the chaotropic agent is NaSCN and the concentration of NaSCN in the liquid lysis composition lies in the range of 0.7M to 1.75M, e.g. 0.75M to 1.5M or preferably 0.8 to 1.25M;

The lysis system may further comprise at least one phosphate. The phosphate is preferably comprised in the lysis composition which comprises the at least one chaotropic agent. According to one embodiment, the phosphate has one or more of the following characteristics:

(i) it is a phosphate dibasic,
(ii) the cationic moiety in the phosphate is selected from ammonium, sodium, potassium, or lithium,
(iii) it is sodium phosphate dibasic.

The concentration of the at least one phosphate in the liquid lysis composition may be from 0.05 to 0.75M. In one embodiment, the concentration of the at least one phosphate in the liquid lysis composition is selected from 0.05 to 0.75M, 0.06M to 0.6M, 0.075M to 0.5M, 0.1M to 0.3M and preferably 0.1 to 0.25M or 0.15M to 0.2M or 0.125M to 0.2M.

In one embodiment, the liquid lysis composition comprises sodium thiocyanate and at least one phosphate, preferably sodium phosphate dibasic.

In one embodiment, the liquid lysis composition comprises sodium thiocyanate in a concentration selected from 0.7M to 1.75M, 0.75M to 1.5M and preferably 0.8 to 1.25M and the at least one phosphate, preferably sodium phosphate dibasic, in a concentration selected from 0.075M to 0.3M, 0.1 to 0.25M and 0.1 M to 0.2M.

In one embodiment, the liquid lysis composition comprises sodium thiocyanate in a concentration from 0.7M to 1.75M and the at least one phosphate, preferably sodium phosphate dibasic, in a concentration from 0.075M to 0.3M, preferably 0.1 to 0.25M and more preferably 0.1M to 0.2M.

The lysis system may further comprise at least one precipitating agent. According to one embodiment, the precipitating agent is selected from ammonium acetate, ammonium sulfate, potassium acetate, sodium acetate, sodium chloride and cesium acetate and wherein preferably, ammonium acetate is used.

The lysis system may further comprise at least one inhibitor removing agent, which is preferably selected from aluminum chloride, aluminum sulfate, erbium (III) acetate, erbium (III) chloride, holmium chloride, zirconium (IV) chloride, hafnium (IV) chloride, aluminum ammonium sulfate, aluminum ammonium sulfate dodecahydrate, aluminum potassium sulfate, aluminum chlorohydrate, calcium oxide, iron (III) chloride, iron (II) sulfate, sodium aluminate, sodium silicate, magnesium chloride, and combinations thereof.

According to one embodiment, at least one inhibitor removing agent is selected from aluminum chloride, erbium (III) acetate, erbium (III) chloride, holmium chloride, hafnium (IV) chloride, zirconium (IV) chloride, guanidine sulfate, and combinations thereof and wherein preferably, the inhibitor removing agent is a trivalent aluminum salt such as aluminum chloride.

According to one embodiment, the lysis system comprises as precipitating agent ammonium acetate, and as inhibitor removing agent a trivalent aluminium salt, preferably aluminum chloride.

According to one embodiment, the precipitating agent and the inhibitor removing agent are comprised in a single composition, preferably a solution, more preferably an aqueous solution. According to one embodiment, the composition has one or more of the following characteristics:

(aa) the total concentration of the one or more precipitating agents in the solution that is added in step (b) is in the range of 0.5 M to 10M, e.g. 1 to 8M, or 1.5 to 7.5M, preferably 1M to 6M, 1.5M to 5.5M, 2M to 5M, 2.5 to 4.5M and 3M to 4M;
(bb) the total concentration of the one or more inhibitor removing agents in the solution that is added in step (b) is in the range of 10 to 500 mM, e.g. 25 mM to 400 mM, 50 mM to 350 mM, 75 mM to 300 mM, 90 mM to 250 mM, preferably 50 mM or 100 mM to 200 mM, such as 50 mM to 175 mM or 75 mM to 150 mM;
(cc) it comprises, consists essentially of, or consists of
(i) one or more precipitating agents selected from ammonium acetate, ammonium sulfate, potassium acetate, sodium acetate, sodium chloride, cesium acetate, and combinations thereof,
(ii) one or more inhibitor removal agents selected from aluminum chloride, erbium (III) acetate, erbium (III) chloride, holmium chloride, hafnium (IV) chloride, zirconium (IV) chloride, and combinations thereof, and
(iii) optionally water.

Further embodiments are also described in conjunction with the method according to the first aspect.

According to one embodiment, (i) the first type is provided by a single solid disrupting particle and (ii) the second type is provided by a plurality of zirconia beads, preferably having a size that lies in the range of 0.08 mm to 0.7 mm, more preferably 0.09 mm to 0.6 mm.

The lysis system may further comprise a nucleic acid-binding solid support.

The lysis system may further comprise one or more of the solutions selected from a DNA binding solution, a DNA wash solution, a DNA elution solution, a RNA binding solution, a RNA wash solution, and a RNA elution solution.

The present invention is also directed to the use of such lysis system which preferably is a kit for lysing plant samples comprising or suspected of comprising microorganisms.

### The Uses According to the Fourth and Fifth Aspects

According to a forth aspect, the present disclosure pertains to the use of the system according to the third aspect in a method according to the first aspect. It is referred to the above disclosure. Suitable plant samples were also described above and it is referred to the above disclosure.

According to a fifth aspect, the present disclosure pertains to the use of the system according to the third aspect for lysing a plant sample and releasing microbial nucleic acids from microorganisms comprised in the plant sample, wherein the user may use (i) the first and the second type or (ii) the second type of disrupting particles for plant sample lysis to release microbial nucleic acids, preferably DNA, from microorganisms comprised in the plant sample. This allows a differential lysis according to the present method.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole. Suitable and preferred embodiments of the aspects of the present invention, such as individual steps and the used components and reagents were described in detail above and as will be appreciated by the skilled person, the disclosure with respect to the individual steps and components and reagents used in the different aspects can be combined with each other. The subject-matter resulting from a respective combination of individual features also belongs to the present disclosure.

The term “solution” as used herein in particular refers to a liquid composition, preferably an aqueous composition. It may be a homogenous mixture of only one phase but it is also within the scope of the present invention that a solution comprises solid constituents, specifically in minor amounts.

Reference to “the disclosure” and “the invention” and the like includes single or multiple aspects taught herein; and so forth. Aspects taught herein are encompassed by the term “invention”.

According to one embodiment, subject matter described herein as comprising certain steps in the case of methods or as comprising certain ingredients in the case of compositions, solutions and/or buffers refers to subject matter consisting of the respective steps or ingredients. It is preferred to select and combine preferred embodiments described herein and the specific subject-matter arising from a respective combination of preferred embodiments also belongs to the present disclosure.

### BRIEF DESCRIPTION OF THE FIGURES

FIG. 1 shows the total DNA yield (in μg) obtained from pine needle samples using different solid disrupting particles and combinations thereof for mechanical disruption of the plant sample.

FIG. 2 shows the total DNA yield (in μg) obtained from root samples using either a ballcone or zirconia beads alone, or a combination thereof for mechanical disruption of the plant sample.

FIG. 3 shows the percentage of microbial reads (FIG. 3a) and bacterial reads (FIG. 3b) obtained from rose leaf samples using either a ballcone or zirconia beads alone, or a combination thereof for mechanical disruption of the plant sample.

FIG. 4 shows the percentage of microbial reads (FIG. 4a) and bacterial reads (FIG. 4b) obtained from maple leaf samples using either a ballcone or zirconia beads alone, or a combination thereof for mechanical disruption of the plant sample.

FIG. 5a shows a rarefaction curve, which is generally used to show the amount on species richness in a sample. FIG. 5a demonstrates that the method where the combination of particles according to the invention was used for lysis resulted in the highest curve, indicating that more bacterial species were detected there than in the other methods. FIG. 5b further supports that in particular with deeper sequencing, the combination will provide more information than the zirconium beads alone.

FIG. 6 shows one embodiment of a disrupting particle, here a ballcone shaped particle. This design combines the burnishing abilities of spheres and cones. Exemplary dimensions of A and B in which ballcone shaped beads can be applied are stated in the following Table II (in inches):

TABLE 11

| Order | Dimensions | |
|---|---|---|
| Size | A | B |
| ⅛″ | .125″ | .170″ |
| 5/32″ | .215″ | .270″ |
| 3/16″ | .270″ | .300″ |
| ¼″ | .320″ | .400″ |
| 5/16″ | .375″ | .465 |

FIGS. 7 and 8 show further exemplary shapes of solid, non-spherical disrupting particles having a surface containing a first part and a second part, whereby the first part and the second part meet by forming an edge, here in the form of sloping central flange. FIG. 7 shows an embodiment with two cone-like tips. FIG. 8 shows an embodiment with two semi-spheres.

FIG. 9 shows the total DNA yield obtained from apple leaf (50 mg) using the lysis chemistry of the present disclosure together with either a ballcone or zirconia beads alone, or a combination thereof for mechanical disruption of the plant sample (Qubit). The results demonstrate that the amount of total DNA (plant and microbial) isolated from apple leaf samples is strongly increased when using a lysis chemistry according to the present disclosure together with zirconia beads and a ballcone for tissue disruption compared to using zirconia beads alone.

FIG. 10 shows the total DNA yield obtained from apple root using the lysis chemistry of the present application with a mixture of a ballcone and zirconia beads or zirconia beads alone. Total DNA quantification was done using Qubit. Furthermore, the yield of microbial DNA, as assessed using a QuantiTect SYBR green assay, is shown. The data demonstrates an improved yield of microbial DNA by combining zirconia beads with a ballcone for the lysis of the plant sample material.

FIG. 11 shows an operational taxonomy unit (OTU) clustering based on the 16S RNA marker gene, which is an operational definition used to classify groups of closely related microbial species. DNA was isolated from apple tree root. Percentages of reads from different microbial species are indicated in the figure. As can be seen, plant cells are more efficiently lysed using a ballcone, whereas zirconia beads are more efficient in lysing bacterial cells. Using a combination combines advantages.

### EXAMPLES

It should be understood that the following examples are for illustrative purpose only and are not to be construed as limiting this invention in any manner.

### I. Materials and Methods

In the following examples, the effectiveness of different solid disrupting particles used as grinding media to support lysis of various plant samples by mechanical disruption was tested. Inter alia, the following disrupting particles and combinations of disrupting particles were tested:

(1) Zirconia beads in two different sizes (0.1 mm and 0.5 mm (diameter); 0.75 g each per sample preparation). The zirconia beads were substantially spherical.
(2) Spherical stainless steel beads (approx. 2.4 mm; 3 per sample preparation).
(3) Ballcone. The ballcone had a size in the range of 4 mm to 7 mm and a weight in the range of 600 mg to 900 mg. The ballcone used was made of steel.
(4) Zirconia beads and spherical steal beads (combination of (1) and (2))
(5) Zirconia beads and ballcone (combination of (1) and (3)).

Plant DNA was isolated using the following general protocol below unless otherwise stated:

### 1. Plant Sample Lysis

Up to 50 mg of plant sample of different origins (e.g. pine needles, root, rose leaf, maple leaf) were collected.

The respective samples were each put in a collection tube (tissue disruption tube, QIAGEN) containing 500 μl of a lysis solution. The lysis solution comprised NaSCN and Na₂HPO₄. Preferred concentrations are described herein. E.g. NaSCN can be present in the lysis solution in a concentration lying in a range of 0.8M to 1.25M. Na₂HPO₄ can be present in a concentration lying in a range of 0.1M to 0.25M or 0.15M to 0.2M. Na₂HPO₄ is preferably comprised in the lysis solution but could also be added separately. An according lysis solution was used in the examples below.

Furthermore, the collection tube comprised the disrupting particle(s) or combination of disrupting particles as indicated above (see (1) to (5)) for mechanical disruption. The samples were briefly vortexted to mix and homogenized for lysis by 2 homogenizing cycles (TissueLyzer II, QIAGEN) of 2 minutes each (@24 Hz).

When processing a plant sample high in phenolic compounds (such as pine needles, see Example 1), one may optionally use 450 μl lysis solution and 50 μl of a phenolic substance suppressing (PSS) buffer, comprising PVP.

The lysates were centrifuged at 12,000×g for 2 min to clear the lysate and the supernatant transferred into a clean tube (approx. 350-450 μl). The supernatant may still comprise some plant particles. Centrifugation can be performed in the Tissue Disruption Tube.

### 2. Inhibitor Removal

200 μl of an IRT solution were added to the supernatant and the sample was vortexed briefly for 5 sec. When processing plants rich in phenolic compounds, the PSS buffer could be alternatively added at this step instead of the lysis step. As disclosed herein, the use of such buffer is optional.

The samples were centrifuged at 12,000 g for 1 min at room temperature. Avoiding the pellet, the supernatant (liquid phase) was transferred into a clean tube. The amount of supernatant was approx. 400-500 μl.

The Inhibitor removal solution (IRT) comprised ammonium acetate as precipitating agent and AlCl₃ as inhibitor removing agent. Preferred concentrations for both agents are described herein. E.g. ammonium acetate can be comprised in the IRT solution in a concentration that lies in a range of 3M to 4M. Aluminum chloride can be comprised in the IRT solution in a concentration that lies in a range of 100 mM to 150 mM. An according lysis solution was used in the examples below.

### 3. Isolation of Nucleic Acids

As described herein, essentially any nucleic acid isolation protocol can be used to isolate and hence recover nucleic acids comprised in the obtained liquid phase (supernatant). In the following, a nucleic acid isolation protocol was used for recovering DNA, wherein DNA is bound to a solid silica support in the presence of a chaotropic salt. A commercially available buffer containing a chaotropic agent (buffer AVL, Qiagen) was added in a volume approx. corresponding to the volume of the supernatant. The DNA in the lysate was bound to a silica spin column (e.g. QIAGEN), the sample-containing tubes were centrifuged and the flow through discarded. Two washing steps of the column-bound DNA were performed, before the samples were eluted into an elution buffer (QIAGEN). The following protocol was followed:

Add 500 μl of Solution AVL and vortex for 5 s. Load 650 μl of the lysate onto an MB Spin Column and centrifuge at 12,000×g for 1 min. Discard the flow-through and repeat step to ensure that all of the lysate has passed through the MB Spin Column (MO BIO). Carefully place the MB Spin Column into a clean 2 ml Collection Tube. Avoid splashing any flow-through onto the MB Spin Column.

Add 500 μl of AW1 (wash buffer, QIAGEN) to the MB Spin Column. Centrifuge at 12,000×g for 1 min. Discard the flow-through and place the MB Spin Column back into the same 2 ml Collection Tube. Add 500 μl of AW2 (wash buffer, QIAGEN) to the MB Spin Column. Centrifuge at 12,000×g for 1 min. Discard the flow-through and place the MB Spin Column into the same 2 ml Collection Tube. Centrifuge at up to 16,000×g for 2 min. Carefully place the MB Spin Column into a new 1.5 ml Elution Tube (provided).

Add 50-100 μl of Solution EB (elution buffer, QIAGEN) to the center of the white filter membrane. Centrifuge at 12,000×g for 1 min. Discard the MB Spin Column. The eluate comprises the eluted DNA.

The eluted DNA was then analysed.

### 4. DNA Quantification

Quantification of isolated DNA was done by fluorometric means (Qubit dsDNA, HS or BR assay kit, Invitrogen) using 5 μl eluate obtained from 4 independent replicates, i.e. 4 individual samples processed.

Specific quantification of microbial DNA was also done using the QuantiTect SYBR green assay (QIAGEN) with primers specific for the 16S rRNA gene and a standard reference sample for quantification following the instructions of the manual. 8 μl of the eluate from 4 independent replicates were applied to the assay, whereby each sample was run in triplicates in the assay. The concentration was determined by comparison to a standard curve using linear regression.

### 5. Next Generation Sequencing

400 ng of isolated DNA were used for library construction. Library construction followed manufacturer's instructions for the QIASeq FX DNA library kit. Libraries were sequenced on an Illumina MiSeq and analyzed with CLC Microbial Genomics Workbench. The libraries were mapped against all available bacterial genomes. The percentage of bacterial reads was determined by taking the number of reads mapping to that reference microbial database, divided by the total number of reads in the library.

### II. Results

### 1. DNA Yield

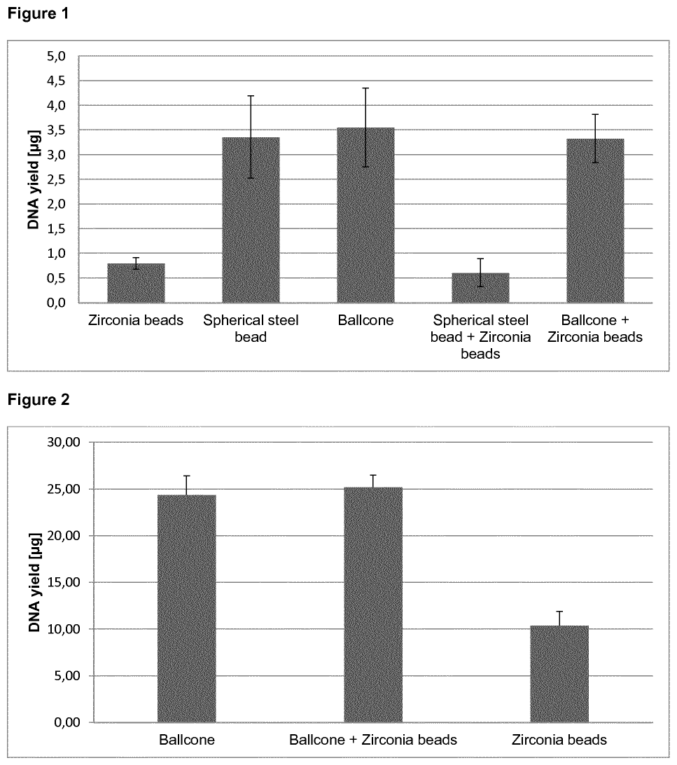

The results are shown in FIG. 1 and FIG. 2. Each column represents the average of 4 independent replicates, the standard deviation is indicated.

### Pine Needle Samples

FIG. 1 shows the DNA yield obtained from pine needle samples (50 mg). As can be seen, the ballcone as a preferred example of an irregularly shaped disrupting particle as used in the present invention provided the highest DNA yield and therefore was most effective in disrupting the plant sample tissue. In contrast, zirconia beads alone only provided very low DNA yields. Therefore, zirconia beads alone do not sufficiently disrupt the plant tissue which is reflected in the reduced DNA yield.

While spherical steel beads alone were effective in lysing the samples, the DNA yield was significantly reduced when using a combination of spherical steel beads and zirconium beads (4). The yield of the combination was even lower than the yield obtained with zirconia beads alone. Thus, the spherical steel particles and the spherical zirconium beads apparently impair each other in the efficiency of mechanical cell lysis. The spherical steel beads might hinder the movement of the zirconia beads around the spherical surface thereby reducing the effectiveness of zirconia bead bashing.

In contrast, the combination of a ballcone with zirconia beads (5) was highly effective in lysing the sample as shown by the high total yield of isolated total DNA. The irregular ballcone shape allows efficient sample mixing and free movement of the zirconia beads, thereby ensuring efficient mechanical plant sample and also microorganism lysis. Therefore, the use of a ballcone as a preferred example of a disrupting particle that is used according to the invention is especially suitable for combination with a plurality of small particles such as zirconia beads. The continued high effectiveness of mechanical lysis of the bacterial cells by zirconium beads in the presence of a ballcone is noteworthy. The combination of the irregular ballcone shape and zirconia beads thus provides very high DNA yields and enables efficient lysis of the plant sample as well as comprised microorganisms such as bacteria and fungi (see also below).

### Root Samples

A high total DNA yield is also obtained when using a combination of a ballcone and zirconium beads for processing difficult to lyse plant samples. This was demonstrated by the processing of root samples. Root is a plant organ especially rich in microorganisms such as bacteria. To efficiently release microbial nucleic acids comprised in root samples it is important to achieve a thorough disruption and lysis of the root sample, because microorganisms such as bacteria may also be comprised inside the root sample. FIG. 2 shows the DNA yield obtained when processing root samples (50 mg).

Zirconia beads alone were not efficient in lysing and homogenizing the root sample as is evident from the reduced DNA yield. This is critical because microorganisms such as bacteria which are inside the plant tissue (here roots) will not be reached by the zirconia beads alone. Therefore, microbial nucleic acids originating from microorganisms that are present inside the plant sample can be lost for analysis when using zirconia beads alone.

In contrast, the combination of a ballcone with zirconia beads provides a high DNA yield thereby indicating that the root sample was efficiently lysed when using the combination of these two types of disrupting particles. The results also show that root samples can be lysed efficiently when using a ballcone alone. However, the ballcone alone is not very efficient for lysing microorganisms comprised in the root sample, i.e. less microbial nucleic acids are released when using a ballcone alone compared to using a combination of a ballcone with zirconia beads (see below).

### Summary

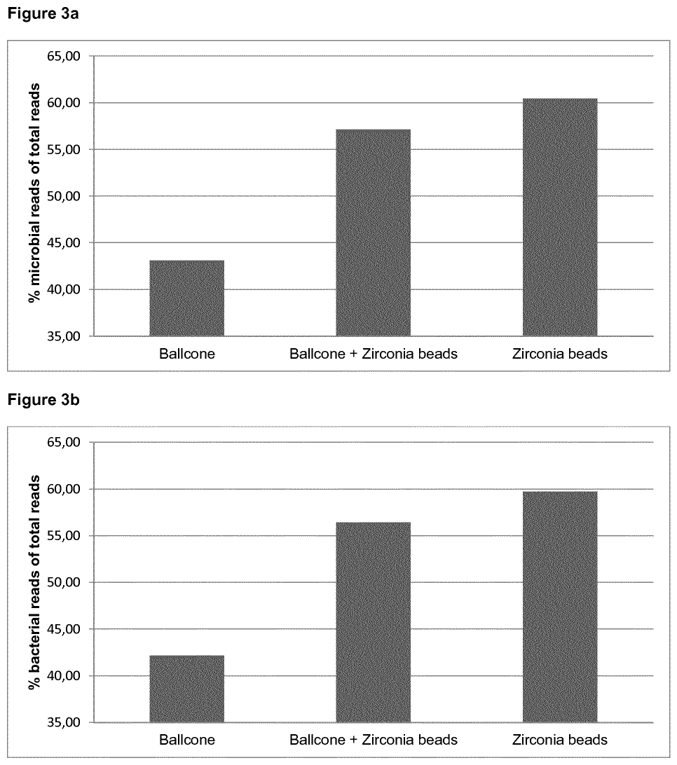

The use of a ballcone as disrupting particle provides high DNA yields and therefore is particularly effective in homogenizing and hence disrupting various plant samples. However, such large disrupting particle alone is less effective in mechanically disrupting microorganisms such as bacteria that are comprised in the plant sample (see FIGS. 3 to 5 discussed below). Large particles are in general not sufficiently effective for lysing microbes.

The combined use of a ballcone together with zirconium beads achieves the same high total yield of isolated total DNA as the use of a ballcone alone. The total DNA yield obtained when using this combination for mechanical lysis was significantly higher compared to the use of zirconium beads alone or a combination of zirconia beads and spherical metal beads in the lysis step. Moreover, the combined use of a ballcone and zirconium beads efficiently releases microbial nucleic acids comprised in microbes comprised in plant samples, as is shown by the high percentage of bacterial DNA among total DNA isolated (see FIGS. 3 to 5 discussed below). Therefore, the combined use of a ballcone together with zirconium beads, especially when being used in conjunction with the lysis chemistry disclosed herein, is preferred. The present invention can be advantageously used to release nucleic acids including microbial nucleic acids from various plant samples, including difficult to lyse samples such as root samples.

### 2. Percentage of Microbial Reads

The use of a ballcone alone, while efficiently lysing various plant samples, is not sufficient to efficiently lyse microorganisms such as bacteria that are comprised in a plant sample. Therefore, microbial nucleic acids are to a certain extent lost when just using a ballcone alone for plant sample lysis.

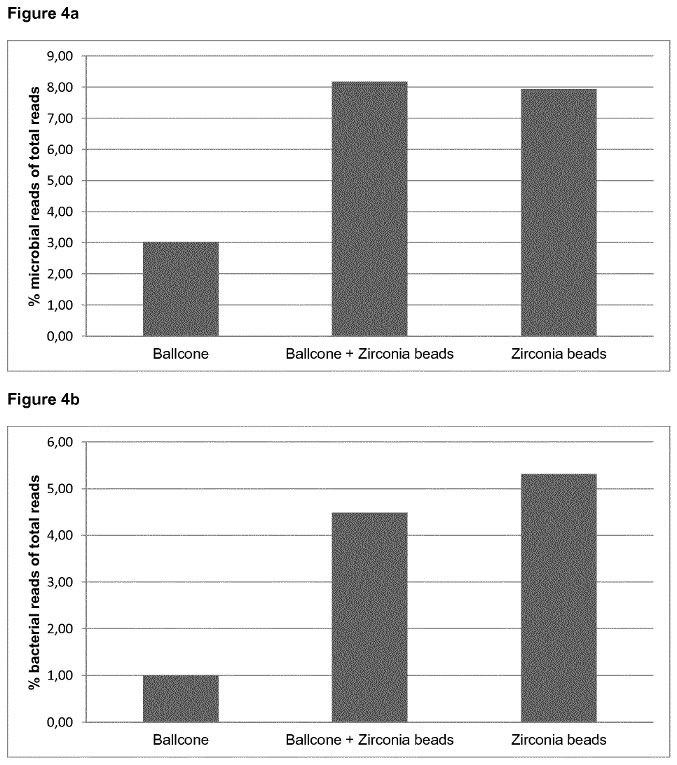

Therefore, for performing a lysis that efficiently releases microbial nucleic acids such as bacteria DNA in addition to the plant DNA, it is advantageous to use a ballcone in combination with zirconia beads. As can be seen from FIGS. 3 and 4, the combination achieves a high percentage of microbial (bacterial and fungal) and also bacterial reads, thereby indicating efficient plant sample lysis as well as efficient microorganism lysis. The higher release of microbial DNA is reflected by the higher percentage of microbial and/or bacterial reads that were obtained in next generation sequencing that followed DNA isolation.

The results demonstrate that using a ballcone in combination with the zirconia beads achieved a higher yield of microbial DNA compared to using a ballcone alone. Furthermore, the results indicate that the overall amount of released and thus recoverable microbial DNA is increased when using a combination of a ballcone with zirconia beads compared to zirconia beads alone. As is shown by FIGS. 1 and 2 discussed above, zirconia beads alone do not efficiently lyse the plant sample, thereby loosing for example to a certain extent microorganisms such as bacteria that are comprised in the plant sample. In this respect it is noted that an increased overall amount of bacterial or microbial DNA in the isolated DNA can nevertheless result in a lower percentage of microbial/bacterial reads, if there is a lot of plant derived DNA.

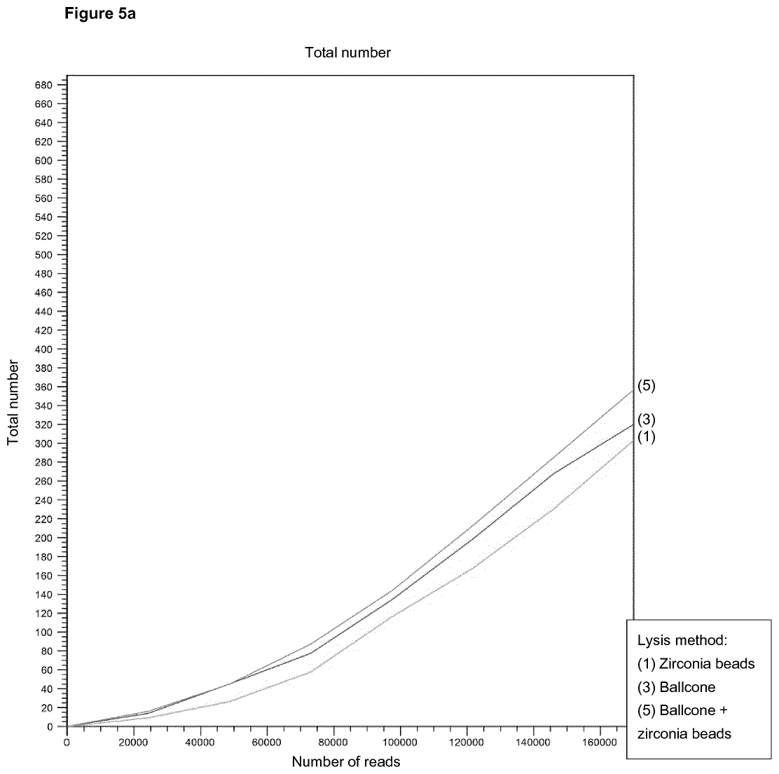

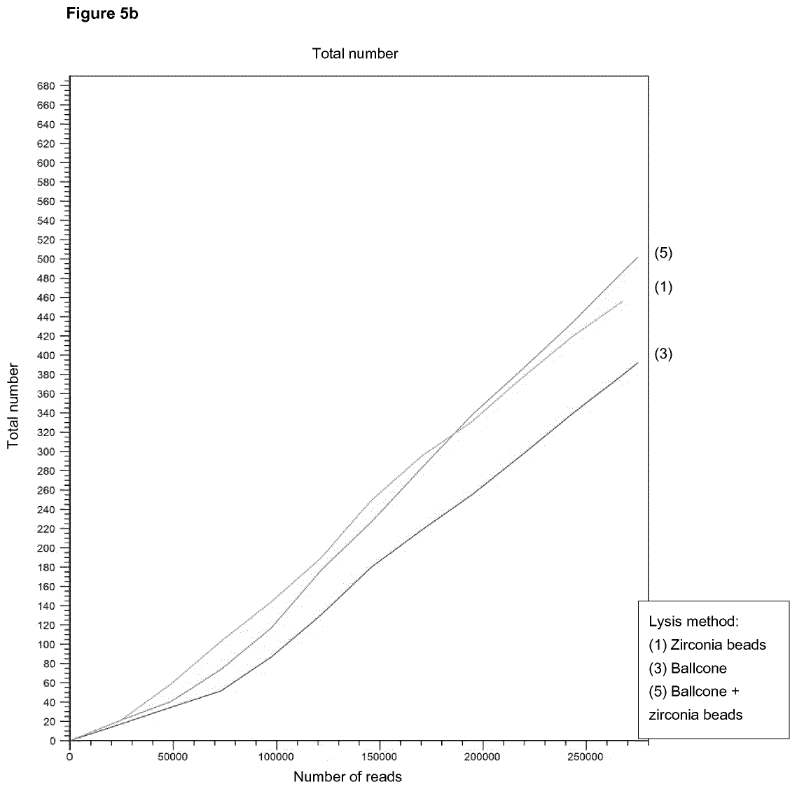

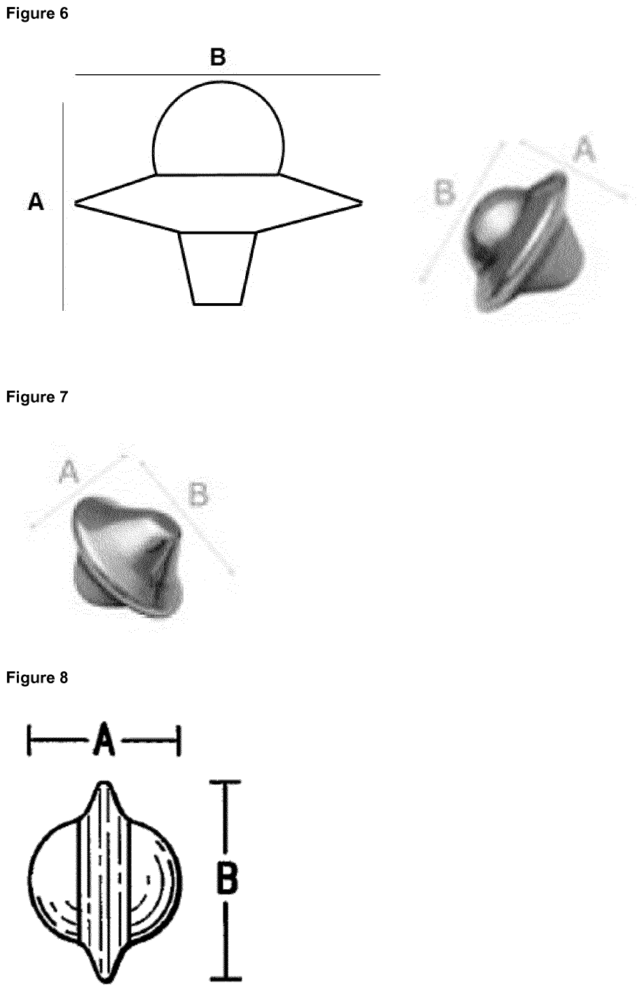

As the combination of particles provides significantly higher total DNA yields and renders accessible microorganisms that are e.g. inside the plant sample (e.g. in case of roots), the total amount of microbial DNA is improved compared to the use of zirconia beads alone. Microbial nucleic acids comprised in the microorganisms contained within the plant sample are additionally released and therefore can be subsequently isolated when using the method of the invention. This is reflected in the sequencing results which showed a higher diversity of 16S sequences for samples were the combination of particles according to the present invention was used for lysis compared to zirconia beads or the ballcone alone (see FIG. 5a).

### Summary

The combined mechanical lysis using a non-spherical disrupting particle as described herein, such as a ballcone, and a plurality of small spherical particles such as zirconium beads provides DNA from plant samples with high yield, wherein the obtained DNA comprises a high amount of microbial DNA which is therefore available for analysis. For analysis, various methods can be used, such as amplification based procedures (for example PCR) as well as sequencing, such as next generation sequencing.

The provided sequencing results furthermore demonstrate a significantly improved percentage of obtained microbial such as bacterial reads compared to the sole use of a ballcone. The percentage of bacterial reads essentially corresponds to the use of zirconium beads alone, while, however, the overall DNA yield is improved (see above). This achieved high percentage of bacterial DNA among total DNA obtained by using a solid non-spherical disrupting particle such as a ballcone in combination with zirconium beads for mechanical lysis is important, as bacteria which are inside the plant cells will not be reached efficiently by the zirconia beads alone, because zirconia beads alone do not sufficiently disrupt the plant cells (see above). Hence, the combined use of a ballcone together with zirconium beads, especially when using the lysis chemistry disclosed herein, is highly advantageous.

### 3. Total and Microbial DNA Yield

The effect when using either a ballcone or zirconia beads alone or in combination (see I. Material and Methods) was analysed with further samples.

### Apple Leaf and Apple Root Samples

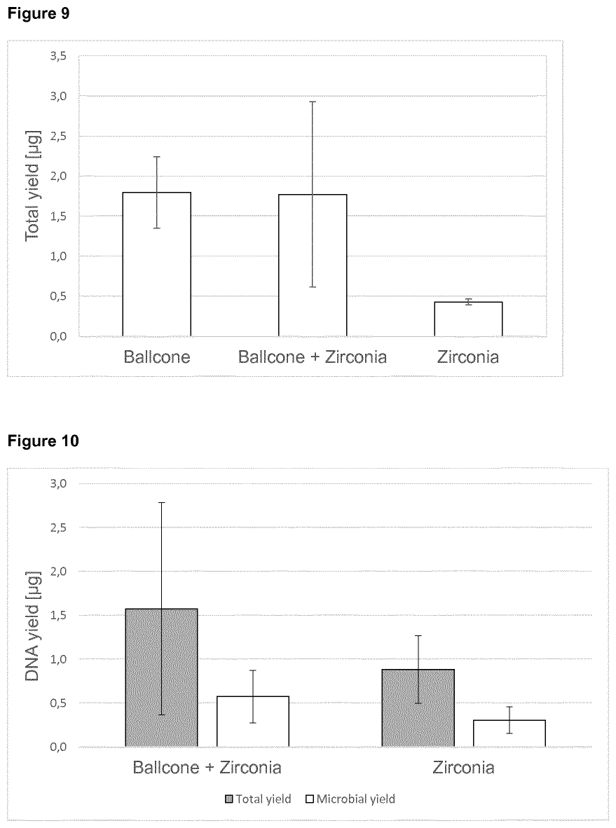

DNA was isolated from apple leaf samples (50 mg) using the lysis method according to the invention in combination with mechanical lysis provided by a ballcone, a ballcone and zirconia beads or zirconia beads alone. Total DNA yield was determined using the Qubit assay. The results shown in FIG. 9 demonstrate that the total DNA yield (plant and microbial) can be increased when using a ballcone and zirconia, compared to zirconia alone.

For FIG. 10, the yields for total (Qubit) and microbial DNA for apple root samples were determined using two different assays. The improved yield of microbial DNA from microbes contained in apple root samples by a combination of a ballcone and zirconia beads over zirconia beads alone is demonstrated in FIG. 10. Microbial DNA was determined using a QuantiTect-based qPCR assay. These data highlight the thorough disruption and lysis of the apple root sample as well as the microbes contained inside the sample which in some cases may strongly increase the overall yield. The data demonstrates the improved yield of microbial DNA by a mixture of a ballcone and zirconia enabling the efficient release of intracellular microbes from the sample over the use of zirconia beads alone.

### Summary

High yields of DNA comprising a high amount of microbial DNA is released from plant samples by a combined mechanical lysis using a non-spherical disrupting particle as described herein, such as a ballcone, and a plurality of small spherical particles such as zirconia beads. For analysis, various methods can be used, such as amplification based procedures (for example PCR) as well as sequencing, such as next generation sequencing, further enabling the amplification-based quantification of microbial DNA contained in the sample.

### 4. The Root-Associated Plant Microbiome

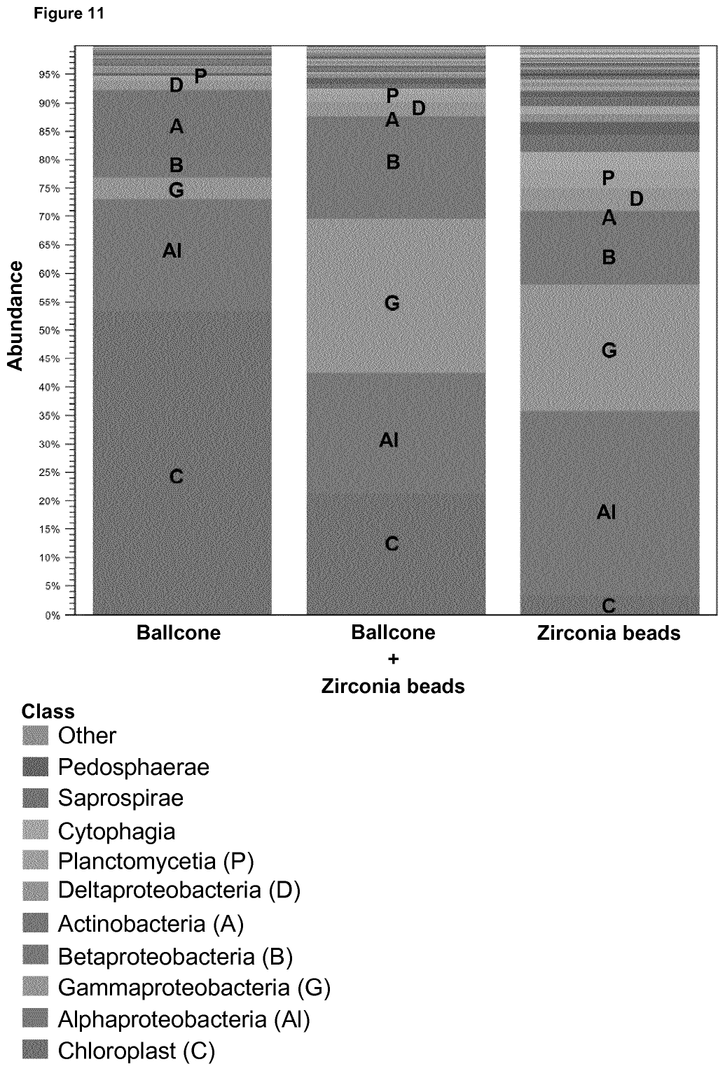

To study the diversity of the microbiome contained within a plant sample and the disruption efficiency of plant associated bacteria, DNA was isolated from 50 mg apple tree root using the method of the invention with a ballcone, a mix of ballcone and small zirconia beads or zirconia beads alone (see Example IV). A 16S rRNA gene library was prepared with the QIAseq FX DNA Library Kit, sequenced by Illumina MiSeq system (2×250 bp run) and the resulting reads were analyzed with the CLC Genomic Workbench (QIAGEN Microbial Genomics Pro Suite). From the results, an operational taxonomy unit (OTU) clustering was performed (FIG. 11). The results demonstrate that plant cells are more efficiently lysed using the ballcone whereas the small zirconia beads are more efficient lysing bacterial cells. Using a combination of a ballcone and zirconia beads has important advantages for certain applications. The combined mechanical lysis using a non-spherical disrupting particle as described herein, such as a ballcone, and a plurality of small spherical particles such as zirconia beads provides DNA from plant samples with high yield, wherein the obtained DNA comprises a high amount of microbial DNA and high microbial diversity.

1. A lysis method for releasing microbial nucleic acids from microorganisms comprised in a plant sample, comprising mechanically disrupting the plant sample in a liquid lysis composition using at least two types of solid disrupting particles, wherein
(i) the first type is provided by one or more disrupting particles having a size of at least 1.5 mm and
(ii) the second type is provided by a plurality of disrupting particles having a size of 1 mm or less.

2. The method according to claim 1, wherein the first type and the second type of solid disrupting particles differ from each other in shape and/or material and wherein preferably, the first type is not spherical and has at least one discontinuity, preferably an edge, and the second type is provided by a plurality of particles that are substantially spherical.

3. The method according to claim 1 or 2, wherein the first type is provided by one or more non-spherical disrupting particles, wherein the surface of the one or more disrupting particles contains a first part and contains a second part, whereby the first part and the second part meet by forming an edge.

4. The method according to claim 3, wherein the first type is provided by one or more non-spherical disrupting particles having one or more of the following characteristics:
(i) the first part is the surface of a frustum of a cone and the second part is the surface of a frustum of a cone, wherein both cones are set against each other with their larger base, the edge being formed where the larger bases meet, the larger basis preferably being of the same diameter;
(ii) the disrupting particle has a subportion that is made up of a section or a part of a ball or an ellipse;
(iii) the disrupting particle has at least one tip, which preferably is a frustum of a cone;
(iv) the disrupting particle has at least two subportions that are made up of a section or a part of a ball or an ellipse;
(v) the one or more disrupting particles have a shape selected from cones, cylinders, cubes, triangles, rectangles, a ballcone and a satellite.

5. The method according to claim 3 or 4, wherein the first type is selected from the following group of particles that are characterized in that:
(aa) the particle comprises at least one tip which is a frustum of a cone, wherein the larger base of the frustum of the cone that provides the tip is set against the smaller base of the frustum of the cone of the second part and wherein the particle comprises a subportion that is made up of a section or a part of a ball or an ellipse which is set against the smaller base of the frustum of the cone of the first part, wherein preferably, the subportion that is made up of a section or a part of a ball or an ellipse is a semi-sphere;
(bb) the particle comprises at least two tips, wherein both tips are a frustum of a cone, wherein the larger base of the frustum of a cone is set against the smaller base of the frustum of the cone of the first part and the larger base of the frustum of a cone is set against the smaller base of the frustum of the cone of the second part;
(cc) the particle comprises two subportions, wherein each subportion is made up of a section or a part of a ball or an ellipse, wherein the first subportion that is made up of a section or a part of a ball or an ellipse is set against the smaller base of the frustum of the cone of the first part and the second subportion that is made up of a section or a part of a ball or an ellipse is set against the smaller base of the frustum of the cone of the second part;
(dd) the particle comprises two semi-spheres wherein the first semi-sphere is set against the smaller base of the frustum of the cone of the first part and the second semi-sphere is set against the smaller base of the frustum of the cone of the second part.

6. The method according to one or more of claims 1 to 5, wherein the first type is provided by one or more non-spherical disrupting particles having a weight in the range of 500 mg to 1000 mg, optionally 600 mg to 900 mg and exhibiting a size of 3 mm to 10 mm, optionally 3 mm to 7 mm or 4 mm to 7 mm.

7. The method according to one or more of claims 1 to 6, wherein the first type is provided by a single solid disrupting particle, preferably as defined in any one of claims 3 to 6, preferably claim 5 or 6.

8. The lysis method according to one or more of claims 1 to 7, wherein the second type has one or more of the following characteristics:
(i) the plurality of particles are crystalline particles;
(ii) the plurality of particles comprise or consist of zirconium, zircon (zirconium silicate), zirconia (zirconium dioxide), yttrium-stabilized zirconium, quartz, aluminum oxide, silicon carbide, ceramic, glasses (e.g. silicon dioxide glass or silica) or a combination of the foregoing;
(iii) the plurality of particles are substantially spherical;
(iv) the plurality of particles have a size that lies in the range selected from 0.05 mm to 0.9 mm, 0.07 mm to 0.8 mm, 0.08 mm to 0.75 mm and 0.09 mm to 0.7 mm;
(v) the plurality of particles are substantially spherical and comprise or consist of zirconium, zircon (zirconium silicate), zirconia (zirconium dioxide) or yttrium-stabilized zirconium and have on average a size that lies in the range of 0.08 mm to 0.7 mm, preferably 0.09 mm to 0.6 mm, wherein preferably, zirconium beads are used;
(vi) the plurality of particles have a density of at least 2.0 g/cc, at least 2.5 g/cc, at least 3.0 g/cc, at least 3.5 g/cc, at least 4.0 g/cc, at least 4.5 g/cc, at least 5.0 g/cc or at least 5.5 g/cc;
(vii) the plurality of particles have a density that lies in a range selected from 2.0 g/cc to 15 g/cc, 2.5 g/cc to 12 g/cc, 3.0 g/cc to 10 g/cc, 3.5 g/cc to 9 g/cc, 4.0 g/cc to 8 g/cc, 4.5 g/cc to 7.5 g/cc and 5 g/cc to 7 g/cc;
(viii) the plurality of particles have at least two different sizes, wherein (i) the first particle size lies on average in a range selected from 0.05 mm to 0.25 mm, 0.07 mm to 0.2 mm, 0.08 mm to 0.175 mm and 0.9 mm to 0.15 mm and (ii) the second particle size lies on average in a range selected from 0.3 mm to 0.9 mm, 0.35 mm to 0.8 mm, 0.4 mm to 0.7 mm and 0.45 mm to 0.6 mm.

9. The lysis method according to one or more of claims 1 to 8, wherein (i) the first type is provided by a single solid disrupting particle as defined in any one of claims 3 to 6, preferably claim 5 or 6, wherein preferably, the single disrupting particle is a ballcone and (ii) the second type is provided by a plurality of substantially spherical zirconia beads, preferably having a size that lies in the range of 0.08 mm to 0.7 mm, more preferably 0.09 mm to 0.6 mm.

10. The lysis method according to one or more of claims 1 to 9, wherein disruption with the first and second type of disrupting particles is performed sequentially or simultaneously, preferably simultaneously.

11. The lysis method according to one or more of claims 1 to 10, wherein the liquid lysis composition comprises at least one chaotropic agent.

12. The lysis method according to claim 11, wherein (i) the chaotropic agent is selected from sodium thiocyanate, potassium thiocyanate, ammonium thiocyanate, lithium thiocyanate and combinations thereof, wherein preferably the chaotropic agent is sodium thiocyanate; and/or (ii) the concentration of the at least one chaotropic agent in the liquid lysis composition and/or the lysis mixture lies in a range of 0.75M to 1.5M and preferably 0.8 to 1.25M, wherein preferably, the chaotropic agent is NaSCN.

13. The lysis method according to one or more of claims 1 to 12, further comprising
clearing the lysate.

14. The lysis method according to one or more of claims 1 to 13, further comprising
contacting the lysed sample, which is optionally cleared, with at least one protein precipitating agent and at least one inhibitor removing agent and providing a mixture; and
obtaining a liquid phase from the mixture;
optionally isolating nucleic acids, preferably DNA, from the liquid phase.

15. The lysis method according to claim 14, having one or more of the following characteristics:
(i) the precipitating agent is selected from ammonium acetate, ammonium sulfate, potassium acetate, sodium acetate, sodium chloride and cesium acetate, wherein preferably, ammonium acetate is used and/or wherein the concentration of the at least one precipitating agent in the mixture is in a range selected from 0.1 to 4M, 0.2M to 3M, 0.3M to 2.5M, 0.4M to 2.25M, 0.5M to 2M and 0.6M to 1.75M;
(ii) the at least one inhibitor removing agent is selected from aluminum chloride, erbium (III) acetate, erbium (III) chloride, holmium chloride, hafnium (IV) chloride, zirconium (IV) chloride, guanidine sulfate, and combinations thereof, wherein preferably, the inhibitor removing agent is a trivalent aluminum salt, more preferably aluminum chloride and/or wherein the concentration of the at least one inhibitor removing agent in the mixture is in a range selected from 1 to 150 mM, 5 mM to 125 mM, 10 mM to 100 mM, 15 mM to 75 mM and 20 mM to 65 mM;
(iii) the precipitating agent is ammonium acetate and the inhibitor removing agent is a trivalent aluminum salt, preferably aluminum chloride;
(iv) the precipitating agent and the inhibitor removing agent are comprised in a single composition, preferably a liquid solution, that is contacted with the lysed sample to provide the mixture; and/or
(v) the method comprises adding at least one phosphate prior to contacting the lysed sample with the at least one inhibitor removing agent, wherein preferably, the at least one phosphate is included in the lysis composition and wherein optionally, the phosphate has one or more of the following characteristics:
(aa) it is a phosphate dibasic,
(bb) the cationic moiety in the phosphate is selected from ammonium, sodium, potassium, or lithium,
(cc) it is sodium phosphate dibasic.

16. The lysis method according to one or more of claims 1 to 15, wherein
(aa) the plant sample is selected from leaf, needle, root, stem, seed, fruit and flowers and wherein preferably, the plant sample is a root sample; and/or
(bb) the microorganisms comprised in the plant samples have one or more of the following characteristics:
(i) the microorganisms are selected from bacteria and fungi, such as gram-positive bacteria, gram-negative bacteria, fungus, mold and spores, or a combination of the foregoing;
(ii) the microorganisms are bacteria;
(iii) the microorganisms are present on, around or within the plant sample, and optionally are comprised in root samples, on leaf surfaces and/or lesions or tumors in the plant tissue.

17. The method according to one or more of claims 1 to 16,
wherein the first type of solid disrupting particles is provided by one or more non-spherical disrupting particles and preferably, the second type of solid disrupting particles is provided by a plurality of substantially spherical particles; and
wherein the liquid lysis composition comprises at least one chaotropic agent in a concentration of 1.5M or less, and wherein the chaotropic agent is selected from sodium thiocyanate, potassium thiocyanate, ammonium thiocyanate, lithium thiocyanate and wherein preferably, the chaotropic agent is sodium thiocyanate; and
wherein the method further comprises
clearing the lysate, wherein clearing the lysate comprises separating the lysed mixture that is obtained upon disrupting the plant sample into a solid fraction and a liquid fraction, wherein the liquid fraction is subsequently processed as lysed sample;
contacting the lysed sample with at least one protein precipitating agent and at least one inhibitor removing agent and providing a mixture; and
obtaining a liquid phase from the mixture;
optionally wherein the method further comprises adding at least one phosphate prior to contacting the lysed sample with the at least one inhibitor removing agent.

18. The method according to claim 17, wherein the at least one protein precipitating agent is selected from ammonium acetate, ammonium sulfate, potassium acetate, sodium acetate, sodium chloride and cesium acetate and wherein preferably, the protein precipitating agent is ammonium acetate, and wherein the at least one inhibitor removing agent is selected from aluminum chloride, erbium (III) acetate, erbium (III) chloride, holmium chloride, hafnium (IV) chloride, zirconium (IV) chloride, guanidine sulfate, and combinations thereof and wherein preferably, the inhibitor removing agent is a trivalent aluminum salt such as more preferably aluminum chloride.

19. The method according to one or more of claims 14 to 18, in particular 17 and 18, wherein the liquid lysis composition comprises sodium thiocyanate as chaotropic agent, and wherein the method comprises contacting the lysed sample with ammonium acetate as precipitating agent and a trivalent aluminum salt, preferably aluminium chloride, as an inhibitor removing agent.

20. The method according to one or more of claims 1 to 19, in particular any one of claims 17 to 19, wherein the liquid lysis composition comprises sodium thiocyanate in a concentration of 0.7M to 1.5M and at least one phosphate, preferably sodium phosphate dibasic, in a concentration of 0.075M to 0.3M.

21. The method according to one or more of claims 1 to 20, in particular any one of claims 17 to 19, wherein the liquid lysis composition comprises sodium thiocyanate in a concentration of 0.8 to 1.25M and at least one phosphate, preferably sodium phosphate dibasic, in a concentration of 0.1 to 0.25M.

22. The method according to one or more of claims 14 to 21, in particular any one of claims 17 to 21, wherein the method comprises contacting the lysed sample with ammonium acetate as precipitating agent and a trivalent aluminum salt, preferably aluminium chloride, as an inhibitor removing agent, wherein in the provided mixture the concentration of ammonium acetate lies is a range of 0.5M to 2M and the concentration of the trivalent aluminum salt lies in a range of 15 mM to 75 mM.

23. The method according to one or more of claims 14 to 22, in particular any one of claims 17 to 22 when dependent on claim 14, further comprising isolating nucleic acids, preferably DNA, from the liquid phase.

24. A method for isolating nucleic acids including microbial nucleic acids from a plant sample, comprising
(a) performing the lysis method according to one or more of claims 1 to 23;
(b) isolating nucleic acids from the lysed and optionally further processed sample; and
(c) optionally sequencing isolated nucleic acid, preferably sequencing isolated DNA.

25. A lysis system, preferably a kit, for releasing microbial nucleic acids from microorganisms comprised in a plant sample, comprising
(a) a liquid lysis composition,
(b) at least two types of solid disrupting particles, wherein
(i) the first type is provided by one or more disrupting particles having a size of at least 1.5 mm; and
(ii) the second type is provided by a plurality of disrupting particles having a size of less than 1 mm,
wherein preferably, the first type of solid disrupting particles has one or more of the characteristics as defined in one or more of claims 2 to 4 and the second type of solid disrupting particles has one or more of the characteristics as defined in claim 5 or 6, and wherein the first and second type of disrupting particles are comprised either in separate containers or in the same container, preferably in the same container.

26. The lysis system according to claim 25, wherein the liquid lysis composition comprises at least one phosphate, optionally as defined in claim 15 (v) (aa), (bb) or (cc), and/or is as defined in one or more of claims 11, 12, 17, 20 and 21.

27. The lysis system according to claim 25 or 26, comprising at least one protein precipitating agent and at least one inhibitor removal agent.

28. The lysis system according to claim 27, wherein the at least one protein precipitating agent is selected from ammonium acetate, ammonium sulfate, potassium acetate, sodium acetate, sodium chloride and cesium acetate and wherein preferably, the protein precipitating agent is ammonium acetate, and wherein the at least one inhibitor removing agent is selected from aluminum chloride, erbium (III) acetate, erbium (III) chloride, holmium chloride, hafnium (IV) chloride, zirconium (IV) chloride, guanidine sulfate, and combinations thereof and wherein preferably, the inhibitor removing agent is a trivalent aluminum salt such as more preferably aluminum chloride.

29. The lysis system according to 27 or 28, wherein the liquid lysis composition comprises sodium thiocyanate as chaotropic agent, and wherein the at least one precipitating agent is ammonium acetate and wherein the inhibitor removing agent is a trivalent aluminum salt, preferably aluminium chloride.

30. Use of the system according to any one of claims 25 to 29 in a method according to any one of claims 1 to 24.

31. Use of the system according to any one of claims 25 to 29 for lysing a plant sample and releasing microbial nucleic acids from microorganisms comprised in the plant sample, wherein the user optionally uses (i) the first and the second type or (ii) the second type of disrupting particles for plant sample lysis to release microbial nucleic acids, preferably DNA, from microorganisms comprised in the plant sample.
